(19) United States
(12) Patent Application Publication
(10) Pub. No.: US 2022/0220214 A1
(43) Pub. Date: Jul. 14, 2022

(54) APJ MODULATORS AND USES THEREOF

(71) Applicant: Ab Initio Biotherapeutics, Inc., San Diego, CA (US)

(72) Inventors: Roy Louis MAUTE, San Diego, CA (US); John S. BURG, San Diego, CA (US)

(21) Appl. No.: 17/615,546

(22) PCT Filed: Jun. 18, 2020
(86) PCT No.: PCT/US2020/038442

§ 371 Date: Nov. 30, 2021

Related U.S. Application Data
(60) Provisional application No. 62/863,775, filed on Jun. 19, 2019.

(51) Int. Cl. C07K 16/28; C07K 16/30

(52) U.S. Cl. CPC C07K 16/2869; A61K 2039/505; C07K 16/30

(57) ABSTRACT

Described herein are protein scaffolds comprising an apelin (APJ) receptor binding domain. Described herein are the uses for the protein scaffolds in treating diseases or disorders comprising aberrant APJ receptor signaling.

### CROSS-REFERENCE

This application claims the benefit of U.S. Provisional Application No. 62/863,775 filed Jun. 19, 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 15, 2020, is named 47815-707_601_SL.txt and is 54,720 bytes in size.

### BACKGROUND

The apelin (APJ) receptor is a G protein-coupled receptor important for various biological functions. APJ signaling has been linked to angiogenesis, which plays a role in diseases and disorders including cancer, diabetes, and cardiovascular disease. Accordingly, targeting APJ receptor signaling has therapeutic potential.

### BRIEF SUMMARY

Provided herein, in some embodiments, are protein scaffolds comprising an apelin (APJ) receptor binding domain, wherein the protein scaffold comprises a variable heavy chain (VH) region or VHH region, wherein the VH region or VHH region comprises: a first sequence CAX₁X₂X₃X₄X₅X₆X₇X₈X₉X₁₀X₁₁X₁₂X₁₃X₁₄X₁₅X₁₆X₁₇X₁₈X₁₉X₂₀YW (SEQ ID NO: 89), wherein X₁ is selected from Y, G, S, D, T, R, A, L, V, P, N, F, E, I, W, Q, K, or H; X₂ is selected from Y, G, S, D, T, R, A, L, V, P, N, F, E, I, W, Q, K, or H; X₃ is selected from Y, G, S, D, T, R, A, L, V, P, N, F, E, I, W, Q, K, or H; X₄ is selected from Y, G, S, D, T, R, A, L, V, P, N, F, E, I, W, Q, K, or H; X₅ is selected from Y, G, S, D, T, R, A, L, V, P, N, F, E, I, W, Q, K, or H; X₆ is selected from Y, G, S, D, T, R, A, L, V, P, N, F, E, I, W, Q, K, or H; X₇ is selected from Y, G, S, D, T, R, A, L, V, P, N, F, E, I, W, Q, K, or H; X₈ is either present or absent, if present, is selected from is selected from Y, G, S, D, T, R, A, L, V, P, N, F, E, I, W, Q, K, or H; X₉ is either present or absent, if present, is selected from is selected from Y, G, S, D, T, R, A, L, V, P, N, F, E, I, W, Q, K, or H; X₁₀ is either present or absent, if present, is selected from is selected from Y, G, S, D, T, R, A, L, V, P, N, F, E, I, W, Q, K, or H; X₁₁ is either present or absent, if present, is selected from is selected from Y, G, S, D, T, R, A, L, V, P, N, F, E, I, W, Q, K, or H; X₁₂ is either present or absent, if present, is selected from is selected from Y, G, S, D, T, R, A, L, V, P, N, F, E, I, W, Q, K, or H; X₁₃ is either present or absent, if present, is selected from is selected from Y, G, S, D, T, R, A, L, V, P, N, F, E, I, W, Q, K, or H; X₁₄ is either present or absent, if present, is selected from is selected from Y, G, S, D, T, R, A, L, V, P, N, F, E, I, W, Q, K, or H; X₁₅ is either present or absent, if present, is selected from is selected from Y, G, S, D, T, R, A, L, V, P, N, F, E, I, W, Q, K, or H; X₁₆ is either present or absent, if present, is selected from is selected from Y, G, S, D, T, R, A, L, V, P, N, F, E, I, W, Q, K, or H; X₁₇ is either present or absent, if present, is selected from is selected from Y, G, S, D, T, R, A, L, V, P, N, F, E, I, W, Q, K, or H; X₁₈ is either present or absent, if present, is selected from is selected from Y, G, S, D, T, R, A, L, V, P, N, F, E, I, W, Q, K, or H; X₁₉ is selected from F, H, L, or Y; and X₂₀ is selected from Y, G, S, D, T, R, A, L, V, P, N, F, E, I, W, Q, K, or H. In some embodiments, at least two of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least three of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least four of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, two of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, three of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, four of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least two of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least three of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least four of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, two of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, three of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, four of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, each of X₄ and X₅ is independently an R. In some embodiments, each of X₆, X₇, and X₈ is independently an R. In some embodiments, each of X₂, X₃, and X₄ is independently an R. In some embodiments, each of X₅, X₆, and X₇ is independently an R. In some embodiments, each of X₇, X₈, X₁₄, and X₁₅ is independently an R. In some embodiments, each of X₅ and X₆ is independently an R. In some embodiments, each of X₄, X₅, X₁₁, and X₁₂ is independently an R. In some embodiments, each of X₁₄ and X₁₅ is independently an R. In some embodiments, each of X₃, X₄, and X₅ is independently an R. In some embodiments, each of X₁₃ and X₁₄ is independently an R. In some embodiments, the first sequence is a CDR3 sequence. In some embodiments, the first sequence is selected from SEQ ID NOs: 1-22. In some embodiments, the VH region or VHH region further comprises: a second sequence GX₂₁IX₂₂X₂₃X₂₄X₂₅X₂₆M (SEQ ID NO: 90), wherein X₂₁ is selected from Y, T, S, or N; X₂₂ is selected from F or S; X₂₃ is selected from Y, G, S, D, T, R, A, L, V, P, N, F, E, I, W, Q, K, or H; X₂₄ is selected from Y, G, S, D, T, R, A, L, V, P, N, F, E, I, W, Q, K, or H; X₂₅ is selected from Y, G, S, D, T, R, A, L, V, P, N, F, E, I, W, Q, K, or H; and X₂₆ is selected from Y, G, S, D, T, R, A, L, V, P, N, F, E, I, W, Q, K, or H. In some embodiments, at least two of X₂₃, X₂₄, X₂₅, and X₂₆ is an R. In some embodiments, at least three of X₂₃, X₂₄, X₂₅, and X₂₆ is an R. In some embodiments, at least four of X₂₃, X₂₄, X₂₅, and X₂₆ is an R. In some embodiments, two of X₂₃, X₂₄, X₂₅, and X₂₆ is an R. In some embodiments, three of X₂₃, X₂₄, X₂₅, and X₂₆ is an R. In some embodiments, four of X₂₃, X₂₄, X₂₅, and X₂₆ is an R. In some embodiments, at least two of X₂₃, X₂₄, X₂₅, and X₂₆ are contiguous R's. In some embodiments, at least three of X₂₃, X₂₄, X₂₅, and X₂₆ are contiguous R's. In some embodiments, at least four of X₂₃, X₂₄, X₂₅, and X₂₆ are contiguous R's. In some embodiments, two of X₂₃, X₂₄, X₂₅, and X₂₆ are contiguous R's. In some embodiments, three of X₂₃, X₂₄, X₂₅, and X₂₆ are contiguous R's. In some embodiments, four of X₂₃, X₂₄, X₂₅, and X₂₆ are contiguous R's. In some embodiments, each of X₂₅ and X₂₆ is independently an R. In some embodiments, the second sequence is a CDR1 sequence. In some embodiments, the second sequence is selected from SEQ ID NOs: 23-44. In some embodiments, the VH region or VHH region further comprises: a third sequence EX₂₇VAX₂₈IX₂₉X₃₀GX₃₁X₃₂TX₃₃Y (SEQ ID NO: 91) or EX₃₄VAIX₃₅X₃₆GX₃₇X₃₈TX₃₉Y (SEQ ID NO: 92), wherein X₂₇ is selected from F or L; X₂₈ is selected from A, G, S, or T; X₂₉ is selected from A, D, G, N, S, or T; X₃₀ is selected from Y, G, S, D, T, R, A, L, V, P, N, F, E, I, W, Q, K, or H; X₃₁ is selected from A, G, S, or T; X₃₂ is selected from I, N, S, T; X₃₃ is selected from N or Y, wherein X₃₄ is selected from F or L; X₃₅ is selected from A, G, S, or T; X₃₆ is selected from Y, G, S, D, T, R, A, L, V, P, N, F, E, I, W, Q, K, or H; X₃₇ is selected from A, G, S, or T; X₃₈ is selected from I, N, S, T; and X₃₉ is selected from N or Y. In some embodiments, the third sequence is a CDR2 sequence. In some embodiments, the third sequence is selected from SEQ ID NOs: 45-66. In some embodiments, the protein scaffold blocks APJ receptor ligand binding. In some embodiments, the protein scaffold is an allosteric modulator of the APJ receptor. In some embodiments, the protein scaffold is a negative allosteric modulator of the APJ receptor. In some embodiments, the protein scaffold is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, a nanobody, or ab antigen-binding fragments thereof. In some embodiments, the protein scaffold is a nanobody. In some embodiments, the protein scaffold comprises a sequence selected from SEQ ID NOs: 67-88. In some embodiments, the protein scaffold comprises at least a 40%, 50%, 60%, 70%, 80%, or 90% inhibition of APJ signaling at a concentration range of about 1 nM to about 100 nM. In some embodiments, the protein scaffold comprises at least a 2-fold, 5-fold, 10-fold, 50-fold, 100-fold, 150-fold, 200-fold, or 250-fold inhibition of APJ signaling at a concentration range of about 1 nM (nanomolar) to about 100 nM. In some embodiments, the protein scaffold comprises a Kd less than 200 nM, less than 150 nM, less than 100 nM, less than 75 nM, less than 50 nM, less than 25 nM, or less than 10 nM.

Provided herein, in some embodiments, are pharmaceutical compositions comprising a protein scaffold described herein, and a pharmaceutically acceptable excipient. In some embodiments, the pharmaceutical composition is formulated for systemic administration. In some embodiments, the pharmaceutical composition is formulated for parenteral administration.

Provided herein, in some embodiments, are isolated nucleic acid molecules encoding the protein scaffold described herein.

Provided herein, in some embodiments, are vectors comprising a nucleic acid sequence encoding the protein scaffold as described herein. In some embodiments, the vector is a viral vector. In some embodiments, the viral vector comprises a retrovirus, an adenovirus, an adeno associated virus, a lentivirus, or a herpes virus.

Provided herein, in some embodiments, are host cells producing a protein scaffold described herein.

Provided herein, in some embodiments, are methods of treating a disease or disorder characterized by aberrant APJ signaling in a subject in need thereof, comprising administering to the subject the protein scaffold described herein. In some embodiments, the disease or disorder is diabetes, obesity, cardiovascular disease, cancer, retinopathy, macular degeneration, or fibrosis. In some embodiments, the disease or disorder is diabetes, obesity, cardiovascular disease, or cancer. In some embodiments, the cancer is bladder cancer, brain cancer, breast cancer, bladder cancer, bone cancer, cervical cancer, colorectal cancer, esophageal cancer, head and neck cancer, kidney cancer, liver cancer, lung cancer, ovarian cancer, pancreatic cancer, prostate cancer, skin cancer, stomach cancer, thyroid cancer, or uterine cancer. In some embodiments, the protein scaffold is administered subcutaneous, intraperitoneal, intravenous, intramuscular, or intratumoral. In some embodiments, methods further comprise administering a vascular endothelial growth factor (VEGF) inhibitor. In some embodiments, the VEGF inhibitor is an antibody, an antigen binding fragment, a RNA interfering agent (RNAi), a small interfering RNA (siRNA), a short hairpin RNA (shRNA), a microRNA (miRNA), an antisense oligonucleotide, a peptide, a peptidomimetic, a small molecule, or an aptamer. In some embodiments, the VEGF inhibitor is pazopanib, bevacizumab, sunitinib, cabozantinib, sorafenib, axitinib, regorafenib, ponatinib, cabozantinib, vandetanib, ramucirumab, lenvatinib, aflibercept, or ziv-aflibercept.

Provided herein, in some embodiments, are methods of treating a disease or disorder characterized in a subject in need thereof, comprising administering to the subject a protein scaffold described herein, wherein the subject is resistant to VEGF treatment. In some embodiments, the disease or disorder is diabetes, obesity, cardiovascular disease, cancer, retinopathy, macular degeneration, or fibrosis.

Provided herein are methods of treating a disease or disorder characterized by aberrant APJ signaling in a subject in need thereof, comprising administering to the subject a cell expressing a nucleic acid encoding a protein scaffold as described herein. In some embodiments, the disease or disorder is diabetes, obesity, cardiovascular disease, cancer, retinopathy, macular degeneration, or fibrosis. In some embodiments, the disease or disorder is diabetes, obesity, cardiovascular disease, or cancer. In some embodiments, the cancer is bladder cancer, brain cancer, breast cancer, bladder cancer, bone cancer, cervical cancer, colorectal cancer, esophageal cancer, head and neck cancer, kidney cancer, liver cancer, lung cancer, ovarian cancer, pancreatic cancer, prostate cancer, skin cancer, stomach cancer, thyroid cancer, or uterine cancer. In some embodiments, methods further comprise administering a vascular endothelial growth factor (VEGF) inhibitor. In some embodiments, the VEGF inhibitor is an antibody, an antigen binding fragment, a RNA interfering agent (RNAi), a small interfering RNA (siRNA), a short hairpin RNA (shRNA), a microRNA (miRNA), an antisense oligonucleotide, a peptide, a peptidomimetic, a small molecule, or an aptamer. In some embodiments, the VEGF inhibitor is pazopanib, bevacizumab, sunitinib, cabozantinib, sorafenib, axitinib, regorafenib, ponatinib, cabozantinib, vandetanib, ramucirumab, lenvatinib, bevacizumab, aflibercept, or ziv-aflibercept.

Provided herein, in some embodiments, are methods of treating a disease or disorder characterized by aberrant APJ signaling in a subject in need thereof, comprising administering to the subject a vector as described herein. In some embodiments, the disease or disorder is diabetes, obesity, cardiovascular disease, cancer, retinopathy, macular degeneration, or fibrosis. In some embodiments, the disease or disorder is diabetes, obesity, cardiovascular disease, or cancer. In some embodiments, the cancer is bladder cancer, brain cancer, breast cancer, bladder cancer, bone cancer, cervical cancer, colorectal cancer, esophageal cancer, head and neck cancer, kidney cancer, liver cancer, lung cancer, ovarian cancer, pancreatic cancer, prostate cancer, skin cancer, stomach cancer, thyroid cancer, or uterine cancer. In some embodiments, methods further comprise administering a vascular endothelial growth factor (VEGF) inhibitor. In some embodiments, the VEGF inhibitor is an antibody, an antigen binding fragment, a RNA interfering agent (RNAi), a small interfering RNA (siRNA), a short hairpin RNA (shRNA), a microRNA (miRNA), an antisense oligonucleotide, a peptide, a peptidomimetic, a small molecule, or an aptamer. In some embodiments, the VEGF inhibitor is pazopanib, bevacizumab, sunitinib, cabozantinib, sorafenib, axitinib, regorafenib, ponatinib, cabozantinib, vandetanib, ramucirumab, lenvatinib, aflibercept, or ziv-aflibercept.

Provided herein, in some embodiments, are methods of reducing APJ-mediated angiogenesis in a target cell, comprising: contacting the target cell with a protein scaffold described herein for a time sufficient for binding of the protein scaffold to the APJ receptor, wherein the protein scaffold blocks interaction of the APJ receptor with a ligand of the APJ receptor. In some embodiments, the target cell is a cancer cell. In some embodiments, the cancer cell is from bladder cancer, bone cancer, brain cancer, breast cancer, colorectal cancer, eye cancer, head and neck cancer, kidney cancer, liver cancer, lung cancer, pancreatic cancer, prostate cancer, skin cancer, stomach cancer, thyroid cancer, or uterine cancer. In some embodiments, the target cell is a normal vascular cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic for screening of protein scaffolds comprising an apelin (APJ) receptor binding domain.

FIG. 2 is a graph of cell binding versus bead binding of protein scaffold clones.

FIG. 3 illustrates graphs of cell binding versus bead binding of protein scaffold clones that bind extracellular APJ epitopes.

FIGS. 4A-4B are graphs of cell binding with and without protein scaffolds that compete directly or indirectly with native APJ ligand.

FIG. 4C is a graph illustrating competitive binding of VHH-1on APJ (APJ)-mediated cAMP production in CHO-K1 cells without ligand and with apelin ligand (apelin-13).

FIG. 4D is a graph illustrating competitive binding of VHH-2 on APJ (APJ)-mediated cAMP production in CHO-K1 cells without ligand and with apelin ligand (apelin-13).

FIG. 4E is a graph illustrating competitive binding of VHH-3 on APJ (APJ)-mediated cAMP production in CHO-K1 cells without ligand and with apelin ligand (apelin-13).

FIG. 4F is a graph illustrating competitive binding of VHH-4 on APJ (APJ)-mediated cAMP production in CHO-K1 cells without ligand and with apelin ligand (apelin-13).

FIG. 4G is a graph illustrating competitive binding of VHH-5 on APJ (APJ)-mediated cAMP production in CHO-K1 cells without ligand and with apelin ligand (apelin-13).

FIG. 4H is a graph illustrating competitive binding of VHH-6 on APJ (APJ)-mediated cAMP production in CHO-K1 cells without ligand and with apelin ligand (apelin-13).

FIG. 4I is a graph illustrating competitive binding of VHH-7 on APJ (APJ)-mediated cAMP production in CHO-K1 cells without ligand and with apelin ligand (apelin-13).

FIG. 4J is a graph illustrating competitive binding of VHH-8 on APJ (APJ)-mediated cAMP production in CHO-K1 cells without ligand and with apelin ligand (apelin-13).

FIG. 4K is a graph illustrating competitive binding of VHH-9 on APJ (APJ)-mediated cAMP production in CHO-K1 cells without ligand and with apelin ligand (apelin-13).

FIG. 4L is a graph illustrating competitive binding of VHH-10 on APJ (APJ)-mediated cAMP production in CHO-K1 cells without ligand and with apelin ligand (apelin-13).

FIG. 4M is a graph illustrating competitive binding of VHH-11 on APJ (APJ)-mediated cAMP production in CHO-K1 cells without ligand and with apelin ligand (apelin-13).

FIG. 4N is a graph illustrating competitive binding of VHH-12 on APJ (APJ)-mediated cAMP production in CHO-K1 cells without ligand and with apelin ligand (apelin-13).

FIG. 4O is a graph illustrating competitive binding of VHH-13 on APJ (APJ)-mediated cAMP production in CHO-K1 cells without ligand and with apelin ligand (apelin-13).

FIG. 4P is a graph illustrating competitive binding of VHH-14 on APJ (APJ)-mediated cAMP production in CHO-K1 cells without ligand and with apelin ligand (apelin-13).

FIG. 4Q is a graph illustrating competitive binding of VHH-15 on APJ (APJ)-mediated cAMP production in CHO-K1 cells without ligand and with apelin ligand (apelin-13).

FIG. 4R is a graph illustrating competitive binding of VHH-16 on APJ (APJ)-mediated cAMP production in CHO-K1 cells without ligand and with apelin ligand (apelin-13).

FIG. 4S is a graph illustrating competitive binding of VHH-17 on APJ (APJ)-mediated cAMP production in CHO-K1 cells without ligand and with apelin ligand (apelin-13).

FIG. 4T is a graph illustrating competitive binding of VHH-18 on APJ (APJ)-mediated cAMP production in CHO-K1 cells without ligand and with apelin ligand (apelin-13).

FIG. 4U is a graph illustrating competitive binding of aVHH-19 on APJ (APJ)-mediated cAMP production in CHO-K1 cells without ligand and with apelin ligand (apelin-13).

FIG. 4V is a graph illustrating competitive binding of VHH-20 on APJ (APJ)-mediated cAMP production in CHO-K1 cells without ligand and with apelin ligand (apelin-13).

FIG. 5A is a graph measuring β-arrestin signaling without antibody and with 1 uM of VHH-21.

FIG. 5B is a graph measuring β-arrestin signaling without antibody and with 500 nM of VHH-22.

FIG. 5C is a graph measuring β-arrestin signaling without antibody and with 2 uM of VHH-23.

FIG. 6 illustrates graphs of radiolabeled agonist binding of VHH-21.

FIG. 7 illustrates graphs of β-arrestin recruitment and cAMP signaling without antibody and with VHH-21 (agonist).

FIG. 8 is a graph of VHH-21 binding to the surface of CT26 cells.

FIG. 9A is a graph of CT26 tumor growth in mice following PBS, control Fc-fusion, or VHH-21-Fc treatment.

FIG. 9B is a graph of survival following PBS, control Fc-fusion, or VHH-21-Fc treatment.

FIG. 9C is a graph of angiogenesis following PBS or VHH-21-Fc treatment.

FIG. 10 illustrates binding of fluorescent-labeled recombinant APJ to yeast cells encoding a library of IgG variants, as measured by fluorescence-activated cell sorting (FACS).

FIG. 11 illustrates graphs of nanobody binding and full IgG binding to APJ expressing cells.

FIG. 12 is a graph of β-arrestin signaling by APJ stimulated by a constant concentration of apelin-13, under increasing concentrations VHH-26.

### DETAILED DESCRIPTION

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges, in some embodiments, are independently included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein the term “individual,” “patient,” or “subject” refers to individuals diagnosed with, suspected of being afflicted with, or at-risk of developing at least one disease for which the described compositions and method are useful for treating. In some embodiments, the individual is a mammal. In some embodiments, the mammal is a mouse, rat, rabbit, dog, cat, horse, cow, sheep, pig, goat, llama, alpaca, or yak. In some embodiments, the individual is a human.

### Apelin (APJ) Receptor Protein Scaffolds

The apelin (APJ) receptor is widely distributed in the body and plays a role in various neurological, metabolic, hypertension, respiratory, gastrointestinal, hepatic, kidney, and cancerous diseases or disorders. Specifically, aberrant APJ receptor signaling has been linked to pathological angiogenesis, which is involved in the progression of cancer. Further, subjects treated with inhibitors of angiogenesis such as vascular endothelial growth factor (VEGF) inhibitors develop resistance to such treatments. There exists a therapeutic need to target mediators of angiogenesis such as the APJ receptor.

Described herein are protein scaffolds comprising an apelin (APJ) receptor binding domain. Protein scaffolds as described herein, in some embodiments, comprise motifs of contiguous arginines (“RR” or “RRR”). In some embodiments, the contiguous arginines are highly enriched within complementarity determining regions (CDRs). In some embodiments, the contiguous arginines are highly enriched within CDR1, CDR2, CDR3, or combinations thereof. In some embodiments, the contiguous arginines are highly enriched within CDR3. In some embodiments, the protein scaffolds comprising motifs of contiguous arginines have improved functional activity, structural stability, expression, specificity, or a combination thereof.

In some embodiments, the protein scaffolds comprise a variable heavy chain (VH) region or VHH region, wherein the VH region or VHH region comprises: a first sequence CAX₁X₂X₃X₄X₅X₆X₇X₈X₉X₁₀X₁₁X₁₂X₁₃X₁₄X₁₅X₁₆X₁₇X₁₈X₁₉X₂₀YW (SEQ ID NO: 89), wherein X₁ is selected from Y, G, S, D, T, R, A, L, V, P, N, F, E, I, W, Q, K, or H; X₂ is selected from Y, G, S, D, T, R, A, L, V, P, N, F, E, I, W, Q, K, or H; X₃ is selected from Y, G, S, D, T, R, A, L, V, P, N, F, E, I, W, Q, K, or H; X₄ is selected from Y, G, S, D, T, R, A, L, V, P, N, F, E, I, W, Q, K, or H; X₅ is selected from Y, G, S, D, T, R, A, L, V, P, N, F, E, I, W, Q, K, or H; X₆ is selected from Y, G, S, D, T, R, A, L, V, P, N, F, E, I, W, Q, K, or H; X₇ is selected from Y, G, S, D, T, R, A, L, V, P, N, F, E, I, W, Q, K, or H; X₈ is either present or absent, if present, is selected from is selected from Y, G, S, D, T, R, A, L, V, P, N, F, E, I, W, Q, K, or H; X₉ is either present or absent, if present, is selected from is selected from Y, G, S, D, T, R, A, L, V, P, N, F, E, I, W, Q, K, or H; X₁₀ is either present or absent, if present, is selected from is selected from Y, G, S, D, T, R, A, L, V, P, N, F, E, I, W, Q, K, or H; X₁₁ is either present or absent, if present, is selected from is selected from Y, G, S, D, T, R, A, L, V, P, N, F, E, I, W, Q, K, or H; X₁₂ is either present or absent, if present, is selected from is selected from Y, G, S, D, T, R, A, L, V, P, N, F, E, I, W, Q, K, or H; X₁₃ is either present or absent, if present, is selected from is selected from Y, G, S, D, T, R, A, L, V, P, N, F, E, I, W, Q, K, or H; X₁₄ is either present or absent, if present, is selected from is selected from Y, G, S, D, T, R, A, L, V, P, N, F, E, I, W, Q, K, or H; X₁₅ is either present or absent, if present, is selected from is selected from Y, G, S, D, T, R, A, L, V, P, N, F, E, I, W, Q, K, or H; X₁₆ is either present or absent, if present, is selected from is selected from Y, G, S, D, T, R, A, L, V, P, N, F, E, I, W, Q, K, or H; X₁₇ is either present or absent, if present, is selected from is selected from Y, G, S, D, T, R, A, L, V, P, N, F, E, I, W, Q, K, or H; X₁₈ is either present or absent, if present, is selected from is selected from Y, G, S, D, T, R, A, L, V, P, N, F, E, I, W, Q, K, or H; X₁₉ is selected from F, H, L, or Y; and X₂₀ is selected from Y, G, S, D, T, R, A, L, V, P, N, F, E, I, W, Q, K, or H.

In some embodiments, the first sequence comprises one or more arginine (R) residues. In some embodiments, at least two of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least three of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least four of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least five of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least six of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least seven of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least eight of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least nine of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least ten of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least eleven of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least twelve of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least thirteen of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least fourteen of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least fifteen of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least sixteen of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, at least seventeen of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R. In some embodiments, each of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R.

In some embodiments, the one or more arginine resides of the first sequence are contiguous R's. In some embodiments, at least two of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least three of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least four of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least five of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least six of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least seven of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least eight of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least nine of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least ten of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least eleven of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least twelve of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least thirteen of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least fourteen of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least fifteen of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least sixteen of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, at least seventeen of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's. In some embodiments, each of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's.

In some embodiments, each of X₄ and X₅ is independently an R. In some embodiments, each of X₆, X₇, and X₈ is independently an R. In some embodiments, X₂, X₃, and X₄ is independently an R. In some embodiments, each of X₅, X₆, and X₇ is independently an R. In some embodiments, each of X₇, X₈, X₁₄, and X₁₅ is independently an R. In some embodiments, each of X₅ and X₆ is independently an R. In some embodiments, each of X₄, X₅, X₁₁, and X₁₂ is independently an R. In some embodiments, X₁₄ and X₁₅ is independently an R. In some embodiments, X₃, X₄, and X₅ is independently an R. In some embodiments, X₁₃ and X₁₄ is independently an R.

In some embodiments, the first sequence comprises a CDR3 sequence. In some instances, the CDR3 sequence is arranged within a VH or VHH sequence as follows: Framework sequence-CDR1-Framework sequence-CDR2-Framework sequence-CDR3-Framework sequence.

In some embodiments, the first sequence is selected from Table 1.

TABLE 1

| SEQ ID NO | Sequence |
|---|---|
| 1 | CAASQ [symbol] AYAARIDLFSYHTYW |
| 2 | CAARLSE [symbol] LEGYWSAVHVYW |
| 3 | CAASCRAYASRIDLFSYHTYW |
| 4 | CAAYRKSYTLDTGKRAYTYW |
| 5 | CAV [symbol] YFLDPNTRHVYW |
| 6 | CAANRQLRGLYLERYRYRTYLYW |
| 7 | CAVRGG [symbol] TLQGGSYHLYW |
| 8 | CAVYIADLYVRSYHSYW |
| 9 | CAVYAQRLTYIASARYHRYW |
| 10 | CAVRSPFV [symbol] LDELG [symbol] YHVYW |
| 11 | CAVQYG [symbol] SSLDGGRYNQHTYW |
| 12 | CAVYG [symbol] ISIES [symbol] GVFSYW |
| 13 | CAVSYRGYARGELGG [symbol] YQYW |
| 14 | CAARGRAYSVDVAVAYHTYW |
| 15 | CAAW [symbol] IDYHVRVSRLNYW |
| 16 | CAARYVPYTRQIRYARYVYW |
| 17 | CAADKK [symbol] SLLNYLHFRYW |
| 18 | CAVVGRYRSYDITSYRSTYHYW |
| 19 | CAVNVLRFQPPDGARKLRYW |
| 20 | CAVPDGYRPHLEPD [symbol] GYSAYIYW |
| 21 | CAVRGGIESIYSFRYW |
| 22 | CAATTQRSKHGLRLRALLYW |

In some embodiments, the first sequence is selected from SEQ ID NOs: 1-22. In some embodiments, the first sequence comprises about 80%, 85%, 90%, 95%, 96% 97%, 98%, 99%, or 100% sequence identity to SEQ ID NOs: 1-22.

Described herein are protein scaffolds comprising an apelin (APJ) receptor binding domain comprise a variable heavy chain (VH) region or VHH region, wherein the VH region or VHH region comprises a first sequence as described herein and a second sequence. In some embodiments, the second sequence comprises GX₂₁IX₂₂X₂₃X₂₄X₂₅X₂₆M (SEQ ID NO: 90), wherein X₂₁ is selected from Y, T, S, or N; X₂₂ is selected from F or S; X₂₃ is selected from Y, G, S, D, T, R, A, L, V, P, N, F, E, I, W, Q, K, or H; X₂₄ is selected from Y, G, S, D, T, R, A, L, V, P, N, F, E, I, W, Q, K, or H; X₂₅ is selected from Y, G, S, D, T, R, A, L, V, P, N, F, E, I, W, Q, K, or H; and X₂₆ is selected from Y, G, S, D, T, R, A, L, V, P, N, F, E, I, W, Q, K, or H.

In some embodiments, the second sequence comprises one or more arginine (R) residues. In some embodiments, at least two of X₂₃, X₂₄, X₂₅, and X₂₆ is an R. In some embodiments, at least three of X₂₃, X₂₄, X₂₅, and X₂₆ is an R. In some embodiments, at least four of X₂₃, X₂₄, X₂₅, and X₂₆ is an R.

In some embodiments, the one or more arginine resides of the second sequence are contiguous R's. In some embodiments, at least two of X₂₃, X₂₄, X₂₅, and X₂₆ are contiguous R's. In some embodiments, at least three of X₂₃, X₂₄, X₂₅, and X₂₆ are contiguous R's. In some embodiments, at least four of X₂₃, X₂₄, X₂₅, and X₂₆ are contiguous R's. In some embodiments, two of X₂₃, X₂₄, X₂₅, and X₂₆ are contiguous R's. In some embodiments, three of X₂₃, X₂₄, X₂₅, and X₂₆ are contiguous R's. In some embodiments, four of X₂₃, X₂₄, X₂₅, and X₂₆ are contiguous R's.

In some embodiments, each of X₂₅ and X₂₆ is independently an R.

In some embodiments, the second sequence is a CDR1 sequence. In some instances, the CDR1 sequence is arranged within a VH or VHH sequence as follows: Framework sequence-CDR1-Framework sequence-CDR2-Framework sequence-CDR3-Framework sequence.

In some embodiments, the second sequence is selected from Table 2.

TABLE 2

| SEQ ID NO | Sequence |
|---|---|
| 23 | GSISVGYLM |
| 24 | GNIFRGRVM |
| 25 | GNISPFRAM |
| 26 | GTIFHRGYM |
| 27 | GTISSYVYM |
| 28 | GSISDYFM |
| 29 | GTISTLRTM |
| 30 | GTISLHRYM |
| 31 | GSIFYSRTM |
| 32 | GYISYTSYM |
| 33 | GSIFSTWRM |
| 34 | GYISNYYYM |
| 35 | GSISYNAYM |
| 36 | GTIFGYRYM |
| 37 | GTISYWTM |
| 38 | GSIFAKATM |
| 39 | GYISAVRTM |
| 40 | GTISRSAYM |
| 41 | GTISAAAYM |
| 42 | GSIFRY [symbol] M |
| 43 | GYISPYRYM |
| 44 | GTISYTTRM |

In some embodiments, the second sequence is selected from SEQ ID NOs: 23-44. In some embodiments, the second sequence comprises about 80%, 85%, 90%, 95%, 96% 97%, 98%, 99%, or 100% sequence identity to SEQ ID NOs: 23-44.

Described herein are protein scaffolds comprising an apelin (APJ) receptor binding domain comprising a variable heavy chain (VH) region or VHH region, wherein the VH region or VHH region comprises a first sequence as described herein and a third sequence. In some embodiments, the third sequence is EX₂₇VAX₂₈IX₂₉X₃₀GX₃₁X₃₂TX₃₃Y (SEQ ID NO: 91) or EX₃₄VAIX₃₅X₃₆GX₃₇X₃₈TX₃₉Y (SEQ ID NO: 92), wherein X₂₇ is selected from F or L; X₂₈ is selected from A, G, S, or T; X₂₉ is selected from A, D, G, N, S, or T; X₃₀ is selected from Y, G, S, D, T, R, A, L, V, P, N, F, E, I, W, Q, K, or H; X₃₁ is selected from A, G, S, or T; X₃₂ is selected from I, N, S, T; X₃₃ is selected from N or Y, wherein X₃₄ is selected from F or L; X₃₅ is selected from A, G, S, or T; X₃₆ is selected from Y, G, S, D, T, R, A, L, V, P, N, F, E, I, W, Q, K, or H; X₃₇ is selected from A, G, S, or T; X₃₈ is selected from I, N, S, T; and X₃₉ is selected from N or Y.

In some embodiments, the third sequence is a CDR2 sequence. In some instances, the CDR2 sequence is arranged within a VH or VHH sequence as follows: Framework sequence-CDR1-Framework sequence-CDR2-Framework sequence-CDR3-Framework sequence.

TABLE 3

| SEQ ID NO | Sequence |
|---|---|
| 45 | ELVAGINYGSNTYY |
| 46 | EFVAGIDYGASTNY |
| 47 | EFVAAINTGAITNY |
| 48 | EFVATITSGSSTNY |
| 49 | ELVAAINRGGTTNY |
| 50 | EFVAGITAGGITNY |
| 51 | EFVASIGNGGSTYY |
| 52 | ELVASINRGGSTYY |
| 53 | EFVATIGLGGITNY |
| 54 | ELVATINQGAITNY |
| 55 | EFVAGIDRGGTTYY |
| 56 | ELVATIGSGGNTYY |
| 57 | EFVASITYGGNTYY |
| 58 | EFVASIGRGTSTNY |
| 59 | ELVASISGGGITYY |
| 60 | ELVATITTGTTTNY |
| 61 | EFVAAISYGSITNY |
| 62 | EFVATIAGGGSTNY |
| 63 | EFVAGIDRGATTYY |
| 64 | EFVASISPGTNTNY |
| 65 | ELVAIGRGGNTYY |
| 66 | EFVAAIAGGGSTNY |

In some embodiments, the third sequence is selected from SEQ ID NOs: 45-66. In some embodiments, the third sequence comprises about 80%, 85%, 90%, 95%, 96% 97%, 98%, 99%, or 100% sequence identity to SEQ ID NOs: 45-66.

In some embodiments, the protein scaffold comprises a VH region or VHH region comprising a sequence from Table 4.

TABLE 4

| SEQ | | |
|---|---|---|
| ID NO | Nanobody | Sequence |
| 67 | VHH-21 | QVQLQESGGGLVQAGGSLRLSCAASGSISVGYLMGWYRQAPGK |
| | | ERELVAGINYGSNTYYADSVKGRFTISRDNAKNTVYLQMNSLKP |
| | | EDTAVYYCAASQ [symbol] AYAARIDLFSYHTYWGQGTQVTVSS |
| 68 | VHH-22 | QVQLQESGGGLVQAGGSLRLSCAASGNIFRGRVMGWYRQAPGK |
| | | EREFVAGIDYGASTNYADSVKGRFTISRDNAKNTVYLQMNSLKP |
| | | EDTAVYYCAARLSE [symbol] LEGYWSAVHVYWGQGTQVTVSS |
| 69 | VHH-23 | QVQLQESGGGLVQAGGSLRLSCAASGNISPFRAMGWYRQAPGK |
| | | EREFVAAINTGAITNYADSVKGRFTISRDNAKNTVYLQMNSLKPE |
| | | DTAVYYCAASCRAYASRIDLFSYHTYWGQGTQVTVSS |
| 70 | VHH-1 | QVQLQESGGGLVQAGGSLRLSCAASGTIFHRGYMGWYRQAPGK |
| | | EREFVATITSGSSTNYADSVKGRFTISRDNAKNTVYLQMNSLKPE |
| | | DTAVYYCAAYRKSYTLDTGKRAYTYWGQGTQVTVSSGSG |
| 71 | VHH-3 | QVQLQESGGGLVQAGGSLRLSCAASGTISSYVYMGWYRQAPGK |
| | | ERELVAAINRGGTTNYADSVKGRFTISRDNAKNTVYLQMNSLKP |
| | | EDTAVYYCAV [symbol] YFLDPNTRHVYWGQGTQVTVSSGSG |
| 72 | VHH-2 | QVQLQESGGGLVQAGGSLRLSCAASGSISDYFMGWYRQAPGKE |
| | | REFVAGITAGGITNYADSVKGRFTISRDNAKNTVYLQMNSLKPED |
| | | TAVYYCAANRQLRGLYLERYRYRTYLYWGQGTQVTVSSGSG |
| 73 | VHH-13 | QVQLQESGGGLVQAGGSLRLSCAASGTISTLRTMGWYRQAPGKE |
| | | REFVASIGNGGSTYYADSVKGRFTISRDNAKNTVYLQMNSLKPE |
| | | DTAVYYCAVRGG [symbol] TLQGGSYHLYWGQGTQVTVSSGSG |
| 74 | VHH-17 | QVQLQESGGGLVQAGGSLRLSCAASGTISLHRYMGWYRQAPGK |
| | | ERELVASINRGGSTYYADSVKGRFTISRDNAKNTVYLQMNSLKP |
| | | EDTAVYYCAVYIADLYVRSYHSYWGQGTQVTVSSGSG |
| 75 | VHH-14 | QVQLQESGGGLVQAGGSLRLSCAASGSIFYSRTMGWYRQAPGKE |
| | | REFVATIGLGGITNYADSVKGRFTISRDNAKNTVYLQMNSLKPED |
| | | TAVYYCAVYAQRLTYIASARYHRYWGQGTQVTVSSGSG |
| 76 | VHH-5 | QVQLQESGGGLVQAGGSLRLSCAASGYISYTSYMGWYRQAPGK |
| | | ERELVATINQGAITNYADSVKGRFTISRDNAKNTVYLQMNSLKPE |
| | | DTAVYYCAVRSPFV [symbol] LDELG [symbol] YHVYWGQGTQVTVSSGSG |
| 77 | VHH-9 | QVQLQESGGGLVQAGGSLRLSCAASGSIFSTWRMGWYRQAPGK |
| | | EREFVAGIDRGGTTYYADSVKGRFTISRDNAKNTVYLQMNSLKP |
| | | EDTAVYYCAVQYG [symbol] SSLDGGRYNQHTYWGQGTQVTVSSGSG |
| 78 | VHH-18 | QVQLQESGGGLVQAGGSLRLSCAASGYISNYYYMGWYRQAPGK |
| | | ERELVATIGSGGNTYYADSVKGRFTISRDNAKNTVYLQMNSLKP |
| | | EDTAVYYCAVYG [symbol] ISIES [symbol] GVFSYWGQGTQVTVSSGSG |
| 79 | VHH-6 | QVQLQESGGGLVQAGGSLRLSCAASGSISYNAYMGWYRQAPGK |
| | | EREFVASITYGGNTYYADSVKGRFTISRDNAKNTVYLQMNSLKP |
| | | EDTAVYYCAVSYRGYARGELGG [symbol] YQYWGQGTQVTVSSGSG |
| 80 | VHH-15 | QVQLQESGGGLVQAGGSLRLSCAASGTIFGYRYMGWYRQAPGK |
| | | EREFVASIGRGTSTNYADSVKGRFTISRDNAKNTVYLQMNSLKPE |
| | | DTAVYYCAARGRAYSVDVAVAYHTYWGQGTQVTVSSGSG |
| 81 | VHH-19 | QVQLQESGGGLVQAGGSLRLSCAASGTISYWTMGWYRQAPGKE |
| | | RELVASISGGGITYYADSVKGRFTISRDNAKNTVYLQMNSLKPED |
| | | TAVYYCAAW [symbol] IDYHVRVSRLNYWGQGTQVTVSSGSG |
| 82 | VHH-16 | QVQLQESGGGLVQAGGSLRLSCAASGSIFAKATMGWYRQAPGK |
| | | ERELVATITTGTTTNYADSVKGRFTISRDNAKNTVYLQMNSLKPE |
| | | DTAVYYCAARYVPYTRQIRYARYVYWGQGTQVTVSSGSG |
| 83 | VHH-20 | QVQLQESGGGLVQAGGSLRLSCAASGYISAVRTMGWYRQAPGK |
| | | EREFVAAISYGSITNYADSVKGRFTISRDNAKNTVYLQMNSLKPE |
| | | DTAVYYCAADKK [symbol] SLLNYLHFRYWGQGTQVTVSSGSG |
| 84 | VHH-10 | QVQLQESGGGLVQAGGSLRLSCAASGTISRSAYMGWYRQAPGK |
| | | EREFVATIAGGGSTNYADSVKGRFTISRDNAKNTVYLQMNSLKP |
| | | EDTAVYYCAVVGRYRSYDITSYRSTYHYWGQGTQVTVSSGSG |
| 85 | VHH-7 | QVQLQESGGGLVQAGGSLRLSCAASGTISAAAYMGWYRQAPGK |
| | | EREFVAGIDRGATTYYADSVKGRFTISRDNAKNTVYLQMNSLKP |
| | | EDTAVYYCAVNVLRFQPPDGARKLRYWGQGTQVTVSSGSG |
| 86 | VHH-11 | QVQLQESGGGLVQAGGSLRLSCAASGSIFRY [symbol] MGWYRQAPGK |
| | | EREFVASISPGTNTNYADSVKGRFTISRDNAKNTVYLQMNSLKPE |
| | | DTAVYYCAVPDGYRPHLEPD [symbol] GYSAYIYWGQGTQVTVSSGSG |
| 87 | VHH-8 | QVQLQESGGGLVQAGGSLRLSCAASGYISPYRYMGWYRQAPGK |
| | | ERELVAIGRGGNTYYADSVKGRFTISRDNAKNTVYLQMNSLKPE |
| | | DTAVYYCAVRGGIESIYSFRYWGQGTQVTVSSGSG |
| 88 | VHH-12 | QVQLQESGGGLVQAGGSLRLSCAASGTISYTTRMGWYRQAPGK |
| | | EREFVAAIAGGGSTNYADSVKGRFTISRDNAKNTVYLQMNSLKP |
| | | EDTAVYYCAATTQRSKHGLRLRALLYWGQGTQVTVSSGSG |

In some embodiments, the protein scaffolds as described herein mediate apelin (APJ) receptor signaling. In some embodiments, the protein scaffolds as described herein block binding of ligand to APJ receptor. In some embodiments, the protein scaffolds as described herein are APJ receptor antagonists. In some embodiments, the protein scaffolds as described herein are APJ receptor allosteric modulators. In some embodiments, the allosteric modulator is a negative allosteric modulator. In some embodiments, the protein scaffolds result in antagonistic or allosteric effects at a concentration of at least or about 1 (nanomolar) nM, 2 nM, 4 nM, 6 nM, 8 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 300 nM, 400 nM, 500 nM, 600 nM, 700 nM, 800 nM, 900 nM, 1000 nM, or more than 1000 nM.

Described herein, in some embodiments, the protein scaffolds result in at least or about a 40%, 50%, 60%, 70%, 80%, or 90% inhibition of APJ signaling at a concentration range of about 1 nM to about 100 nM. In some embodiments, the protein scaffolds result in at least or about a 40%, 50%, 60%, 70%, 80%, or 90% inhibition of APJ signaling at least or about 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 300 nM, 400 nM, 500 nM, 600 nM, 700 nM, 800 nM, 900 nM, 1000 nM, or more than 1000 nM. In some embodiments, the protein scaffolds result in at least or about a 2-fold, 5-fold, 10-fold, 50-fold, 100-fold, 150-fold, 200-fold, or 250-fold inhibition of APJ signaling at a concentration range of about 1 nM (nanomolar) to about 100 nM. In some embodiments, the protein scaffolds result in at least or about a 2-fold, 5-fold, 10-fold, 50-fold, 100-fold, 150-fold, 200-fold, or 250-fold inhibition of APJ signaling at least or about 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 300 nM, 400 nM, 500 nM, 600 nM, 700 nM, 800 nM, 900 nM, 1000 nM, or more than 1000 nM.

Described herein, in some embodiments, are protein scaffolds, wherein the protein scaffolds comprise a Kd less than 200 nM, less than 150 nM, less than 100 nM, less than 75 nM, less than 50 nM, less than 25 nM, or less than 10 nM. In some embodiments, the protein scaffolds comprise a Kd at most 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 300 nM, 400 nM, 500 nM, 600 nM, 700 nM, 800 nM, 900 nM, or 1000 nM.

Described herein are protein scaffolds comprising an apelin (APJ) receptor binding domain. In some embodiments, the protein scaffold is an antibody. In some embodiments, the antibody includes, but are not limited to, monoclonal antibodies, polyclonal antibodies, multispecific antibodies (for example, bispecific antibodies and polyreactive antibodies), and antibody fragments. In some embodiments, the antibodies include antibody-conjugates and molecules comprising the antibodies, such as chimeric molecules. In some embodiments, the antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that, in some embodiments, are present in minor amounts. A polyclonal antibody is a preparation that includes different antibodies of varying sequences that generally are directed against two or more different determinants (epitopes). In some embodiments, the monoclonal antibody comprises a human IgG1 constant region. In some embodiments, the monoclonal antibody comprises a human IgG4 constant region.

The term “antibody” herein is used in the broadest sense and includes polyclonal and monoclonal antibodies, including intact antibodies and functional (antigen-binding) antibody fragments thereof, including fragment antigen binding (Fab) fragments, F(ab′)2fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. In some embodiments, the antibody comprises a human IgG1 constant region. In some embodiments, the antibody comprises a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). In some embodiments, the precise amino acid sequence boundaries of a given CDR or FR are readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme.

The boundaries of a given CDR or FR, in some embodiments, vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain, in some embodiments, is sufficient to confer antigen-binding specificity. In some embodiments, antibodies that bind a particular antigen are isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Described herein are protein scaffolds comprising an apelin (APJ) receptor binding domain, wherein the protein scaffolds comprise antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g. scFv or sFv); and multispecific antibodies formed from antibody fragments. In some embodiments, the antibodies are single-chain antibody fragments comprising a variable heavy chain region and/or a variable light chain region, such as scFvs.

In some embodiments, protein scaffolds are made by various techniques, including but not limited to proteolytic digestion of a protein scaffold as well as production by recombinant host cells. In some embodiments, the protein scaffolds are recombinantly-produced fragments, such as fragments comprising arrangements that do not occur naturally, such as those with two or more protein scaffold regions or chains joined by synthetic linkers, e.g., polypeptide linkers, and/or those that are not produced by enzyme digestion of a naturally-occurring protein scaffold. In some aspects, the protein scaffolds are scFvs.

A “humanized” antibody is an antibody in which all or substantially all CDR amino acid residues are derived from non-human CDRs and all or substantially all FR amino acid residues are derived from human FRs. In some embodiments, a humanized antibody optionally includes at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Described herein are protein scaffolds comprising an apelin (APJ) receptor binding domain, wherein the protein scaffolds are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies, in some embodiments, are prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. In some embodiments, human antibodies are be derived from human antibody libraries, including phage display and cell-free libraries, containing antibody-encoding sequences derived from a human repertoire.

The terms “polypeptide” and “protein” are used interchangeably to refer to a polymer of amino acid residues, and are not limited to a minimum length. Polypeptides, including the provided protein scaffolds, e.g., linkers and binding peptides, in some embodiments, include amino acid residues including natural and/or non-natural amino acid residues. The terms also include post-expression modifications of the polypeptide, for example, glycosylation, sialylation, acetylation, phosphorylation, and the like. In some embodiments, the polypeptides contain modifications with respect to a native or natural sequence, as long as the protein maintains the desired activity. These modifications, in some embodiments, are deliberate, as through site-directed mutagenesis, or are accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. In some embodiments, alignment for purposes of determining percent amino acid sequence identity are achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or in some embodiments, compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which, in some embodiments, is phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, amino acid sequence variants of the protein scaffolds provided herein are contemplated. A variant typically differs from a polypeptide specifically disclosed herein in one or more substitutions, deletions, additions and/or insertions. In some embodiments, variants are naturally occurring or are synthetically generated, for example, by modifying one or more of the above polypeptide sequences of the invention and evaluating one or more biological activities of the polypeptide as described herein and/or using any of a number of known techniques. In some embodiments, amino acid sequence variants of are prepared by introducing appropriate modifications into the nucleotide sequence encoding the antibody, or by peptide synthesis. Such modifications include, for example, deletions from, and/or insertions into and/or substitutions of residues within the amino acid sequences of the antibody. In some embodiments, any combination of deletion, insertion, and substitution are made to arrive at the final construct, provided that the final construct possesses the desired characteristics, e.g., antigen-binding.

In some embodiments, protein scaffold variants having one or more amino acid substitutions are provided. Sites of interest for mutagenesis by substitution include the CDRs and FRs. Amino acid substitutions, in some embodiments, are introduced into a protein scaffold of interest and the products screened for a desired activity, e.g., retained/improved antigen binding, decreased immunogenicity, or improved ADCC or CDC.

In some embodiments, substitutions, insertions, or deletions occur within one or more CDRs, wherein the substitutions, insertions, or deletions do not substantially reduce antibody binding to antigen. For example, conservative substitutions that do not substantially reduce binding affinity are made in CDRs. Such alterations, in some embodiments, are outside of CDR “hotspots”. In some embodiments, the CDR is unaltered.

In some embodiments, alterations (e.g., substitutions) are made in CDRs, e.g., to improve antibody affinity. In some embodiments, alterations are made in CDR encoding codons with a high mutation rate during somatic maturation (See e.g., Chowdhury, Methods Mol. Biol. 207:179-196 (2008)), and the resulting variant is tested for binding affinity. In some embodiments, affinity maturation (e.g., using error-prone PCR, chain shuffling, randomization of CDRs, or oligonucleotide-directed mutagenesis) are used to improve antibody affinity (See e.g., Hoogenboom et al. in Methods in Molecular Biology 178:1-37 (2001)). In some embodiments, CDR residues involved in antigen binding are specifically identified, e.g., using alanine scanning mutagenesis or modeling (See e.g., Cunningham and Wells Science, 244:1081-1085 (1989)). CDR-H3 and CDR-L3 in particular are often targeted. Alternatively, or additionally, a crystal structure of an antigen-antibody complex to identify contact points between the antibody and antigen. In some embodiments, such contact residues and neighboring residues are targeted or eliminated as candidates for substitution. In some embodiments, variants are screened to determine whether the variants contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

In some embodiments, the protein scaffolds are altered to increase or decrease their glycosylation (e.g., by altering the amino acid sequence such that one or more glycosylation sites are created or removed). In some embodiments, a carbohydrate attached to an Fc region of an antibody is altered. Native antibodies from mammalian cells typically comprise a branched, biantennary oligosaccharide attached by an N-linkage to Asn₂₉₇ of the CH2 domain of the Fc region (See e.g., Wright et al. TIBTECH 15:26-32 (1997)). The oligosaccharides, in some embodiments, are various carbohydrates, e.g., mannose, N-acetyl glucosamine (GlcNAc), galactose, sialic acid, fucose attached to a GlcNAc in the stem of the biantennar oligosaccharide structure. In some embodiments, modifications of the oligosaccharide in a protein scaffold are made, for example, to create variants with certain improved properties. In some embodiments, glycosylation variants have improved ADCC and/or CDC function. In some embodiments, variants are provided having a carbohydrate structure that lacks fucose attached (directly or indirectly) to an Fc region. For example, the amount of fucose in such antibody is from 1% to 80%, from 1% to 65%, from 5% to 65% or from 20% to 40%. The amount of fucose is determined by calculating the average amount of fucose within the sugar chain at Asn₂₉₇, relative to the sum of all glycostructures attached to Asn297 (See e.g., WO 08/077546). Asn₂₉₇ refers to the asparagine residue located at about position 297 in the Fc region (EU numbering of Fc region residues; See e.g., Edelman et al. Proc Natl Acad Sci USA. 1969 May; 63(1):78-85). Asn₂₉₇, in some embodiments, are located about ±3 amino acids upstream or downstream of position 297, i.e., between positions 294 and 300, due to minor sequence variations in antibodies. In some embodiments, such fucosylation variants have improved ADCC function (See e.g., Okazaki et al. J. Mol. Biol. 336:1239-1249 (2004); and Yamane-Ohnuki et al. Biotech. Bioeng. 87: 614 (2004)). In some embodiments, cell lines, e.g., knockout cell lines and methods of their use are used to produce defucosylated antibodies, e.g., Lec13 CHO cells deficient in protein fucosylation and alpha-1,6-fucosyltransferase gene (FUT8) knockout CHO cells (See e.g., Ripka et al. Arch. Biochem. Biophys. 249:533-545 (1986); Yamane-Ohnuki et al. Biotech. Bioeng. 87: 614 (2004); Kanda, Y. et al., Biotechnol. Bioeng., 94(4):680-688 (2006)). Other antibody glycosylation variants are also included (See e.g., U.S. Pat. No. 6,602,684).

In some embodiments, one or more amino acid modifications are introduced into the Fc region of a protein scaffold provided herein, thereby generating an Fc region variant. An Fc region herein is a C-terminal region of an immunoglobulin heavy chain that contains at least a portion of the constant region. An Fc region includes native sequence Fc regions and variant Fc regions. In some embodiments, the Fc region variant comprises a human Fc region sequence (e.g., a human IgG1, IgG2, IgG3 or IgG4 Fc region) comprising an amino acid modification (e.g., a substitution) at one or more amino acid positions.

In some embodiments, the protein scaffolds provided herein are variants that possess some but not all effector functions, which make it a desirable candidate for applications in which the half-life of the protein scaffold in vivo is important yet certain effector functions (such as complement and ADCC) are unnecessary or deleterious. In some embodiments, in vitro and/or in vivo cytotoxicity assays are conducted to confirm the reduction/depletion of CDC and/or ADCC activities. For example, Fc receptor (FcR) binding assays are conducted to ensure that the antibody lacks FcγR binding (hence likely lacking ADCC activity), but retains FcRn binding ability. Non-limiting examples of in vitro assays to assess ADCC activity of a molecule of interest is described in U.S. Pat. Nos. 5,500,362 and 5,821,337. In some embodiments, non-radioactive assays methods are employed (e.g., ACTI™ and CytoTox 96® non-radioactive cytotoxicity assays). Useful effector cells for such assays include, but are not limited to, peripheral blood mononuclear cells (PBMC), monocytes, macrophages, and Natural Killer (NK) cells.

Protein scaffolds as described herein, in some embodiments, have increased half-lives and improved binding to the neonatal Fc receptor (FcRn) (See e.g., US 2005/0014934). In some embodiments, such antibodies comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

In some embodiments, it is desirable to create cysteine engineered antibodies, e.g., “thioMAbs,” in which one or more residues of an antibody are substituted with cysteine residues. In some embodiments, the substituted residues occur at accessible sites of the antibody. In some embodiments, reactive thiol groups are positioned at sites for conjugation to other moieties, such as drug moieties or linker drug moieties, to create an immunoconjugate.

In some embodiments, a protein scaffold provided herein is further modified to contain additional nonproteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. In some embodiments, polyethylene glycol propionaldehyde is used in manufacturing due to its stability in water. In some embodiments, the polymer comprises any molecular weight, and is branched or unbranched. In some embodiments, the number of polymers attached to the antibody varies, and if two or more polymers are attached, the two or more polymers are the same or different molecules.

In some embodiments, protein scaffolds as described herein are encoded by a nucleic acid. A nucleic acid is a type of polynucleotide comprising two or more nucleotide bases. In some embodiments, the nucleic acid is a component of a vector that is used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which, in some embodiments, becomes integrated into the chromosomal DNA of the host cell. Another type of vector is an “episomal” vector, e.g., a nucleic acid capable of extra-chromosomal replication. Vectors capable of directing the expression of genes to which the vectors are operatively linked are referred to herein as “expression vectors.” Suitable vectors comprise plasmids, bacterial artificial chromosomes, yeast artificial chromosomes, viral vectors and the like. In some embodiments, the expression vectors regulatory elements such as promoters, enhancers, polyadenylation signals for use in controlling transcription are derived from mammalian, microbial, viral or insect genes. In some embodiments, the ability to replicate in a host, usually conferred by an origin of replication, and a selection gene to facilitate recognition of transformants is additionally be incorporated. In some embodiments, vectors derived from viruses, such as lentiviruses, retroviruses, adenoviruses, adeno-associated viruses, and the like, are employed. In some embodiments, plasmid vectors are linearized for integration into a chromosomal location. In some embodiments, vectors comprise sequences that direct site-specific integration into a defined location or restricted set of sites in the genome (e.g., AttP-AttB recombination). In some embodiments, vectors comprise sequences derived from transposable elements.

As used herein, the terms “homologous,” “homology,” or “percent homology” when used herein to describe to an amino acid sequence or a nucleic acid sequence, relative to a reference sequence, in some embodiments, is determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences, in some embodiments, is determined using the most recent version of BLAST, as of the filing date of this application.

In some embodiments, the nucleic acids encoding the protein scaffolds described herein are used to infect, transfect, transform, or otherwise render a suitable cell transgenic for the nucleic acid, thus enabling the production of antibodies for commercial or therapeutic uses. Standard cell lines and methods for the production of protein scaffolds such as antibodies from a large scale cell culture are known in the art. See e.g., Li et al., “Cell culture processes for monoclonal antibody production.” Mabs. 2010 September-October; 2(5): 466-477. In some embodiments, the cell is a eukaryotic cell. In some embodiments, the eukaryotic cell is a mammalian cell. In some embodiments, the mammalian cell is a Chines Hamster Ovary cell (CHO) cell, an NS0 murine myeloma cell, or a PER.C6® cell. In some embodiments, the nucleic acid encoding the protein scaffold is integrated into a genomic locus of a cell useful for producing protein scaffolds. In some embodiments, described herein is a method of making a protein scaffold comprising culturing a cell comprising a nucleic acid encoding a protein scaffold under conditions in vitro sufficient to allow production and secretion of said protein scaffold.

In some embodiments, described herein, is a master cell bank comprising: (a) a mammalian cell line comprising one or more nucleic acids encoding a protein scaffold described herein integrated at a genomic location; and (b) a cryoprotectant. In some embodiments, the cryoprotectant comprises glycerol. In some embodiments, the master cell bank comprises: (a) a CHO cell line comprising a nucleic acid encoding a protein scaffold with (i) a first sequence having SEQ ID NOs: 1-22, a second sequence having SEQ ID NOs: 23-44, a third sequence having SEQ ID NOs: 45-66, or combinations thereof; and (b) a cryoprotectant. In some embodiments, the cryoprotectant comprises glycerol. In some embodiments, the master cell bank is contained in a suitable vial or container able to withstand freezing by liquid nitrogen.

Also described herein are methods of making protein scaffolds. In some embodiments, such methods comprise incubating a cell or cell-line comprising a nucleic acid encoding the protein scaffold in a cell culture medium under conditions sufficient to allow for expression and secretion of the protein scaffold, and further harvesting the protein scaffold from the cell culture medium. In some embodiments, the harvesting further comprises one or more purification steps to remove live cells, cellular debris, non-antibody proteins or polypeptides, undesired salts, buffers, and medium components. In some embodiments, the additional purification step(s) include centrifugation, ultracentrifugation, dialysis, desalting, protein A, protein G, protein A/G, or protein L purification, and/or ion exchange chromatography.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. In some embodiments, mammalian transient expression vectors include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. In some embodiments, mammalian stable expression vector include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. In some embodiments, bacteria are Acidobacteria, Chloroflexi, Chrysiogenetes, Cyanobacteria, Deferribacteres, Dictyoglomi, Thermodesulfobacteria or Thermotogae. In some embodiments, a bacterial cell is Escherichia coli, Clostridium botulinum, or Coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cell include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Therapeutic Methods

The protein scaffolds described herein, in some embodiments, are useful for the treatment of a disease or disorder. In some embodiments, the disease or disorder is associated with aberrant apelin (APJ) receptor signaling. In some embodiments, the disease or disorder is caused by APJ-mediated angiogenesis.

In some embodiments, the disease or disorder is diabetes, obesity, cardiovascular disease, cancer, retinopathy, macular degeneration, or fibrosis. In some embodiments, the disease or disorder is diabetes, obesity, cardiovascular disease, or cancer.

In some embodiments, the disease or disorder is cancer. In some embodiments, the protein scaffolds are used for cancer treatment. Cancer treatment includes, but is not limited to, reduction of tumor volume, reduction in growth of tumor volume, increase in progression-free survival, or overall life expectancy. In some embodiments, treatment will affect remission of a cancer being treated. In some embodiments, treatment encompasses use as a prophylactic or maintenance dose intended to prevent reoccurrence or progression of a previously treated cancer or tumor.

In some embodiments, the cancer or tumor is a solid cancer or tumor. In some embodiments, the cancer or tumor is a blood cancer or tumor. In some embodiments, the cancer or tumor comprises breast, heart, lung, small intestine, colon, spleen, kidney, bladder, head, neck, ovarian, prostate, brain, pancreatic, skin, bone, bone marrow, blood, thymus, uterine, testicular, or liver tumors. In some embodiments, tumors or cancers which can be treated with the protein scaffolds of the invention comprise adenoma, adenocarcinoma, angiosarcoma, astrocytoma, epithelial carcinoma, germinoma, glioblastoma, glioma, hemangioendothelioma, hemangio sarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In some embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Ewing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VIPpoma, and Wilm's tumor. In some embodiments, the tumor/cancer to be treated with one or more antibodies of the invention comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In some embodiments, the cancer is refractory to other treatment. In some embodiments, the cancer treated is relapsed.

In some embodiments, the protein scaffolds are administered to a subject in need thereof by any route suitable for administration, such as, for example, subcutaneous, intraperitoneal, intravenous, intramuscular, intratumoral, or intracerebral, etc. In some embodiments, the protein scaffolds are administered intravenously. In some embodiments, the protein scaffolds are administered subcutaneously. In some embodiments, the protein scaffolds are administered intratumoral. In some embodiments, the protein scaffolds are administered on a suitable dosage schedule, for example, weekly, twice weekly, monthly, twice monthly, once every two weeks, once every three weeks, or once a month etc. In some embodiments, the protein scaffolds are administered once every three weeks. In some embodiments, the protein scaffolds are administered in any therapeutically effective amount. In some embodiments, the therapeutically acceptable amount is between about 0.1 mg/kg and about 50 mg/kg. In some embodiments, the therapeutically acceptable amount is between about 1 mg/kg and about 40 mg/kg. In some embodiments, the therapeutically acceptable amount is between about 5 mg/kg and about 30 mg/kg. Therapeutically effective amounts include amounts sufficient to ameliorate one or more symptoms associated with the disease or affliction to be treated.

Described herein, in some embodiments, are protein scaffolds for treating a disease or disorder, wherein a vascular endothelial growth factor (VEGF) inhibitor is administered. In some embodiments, the VEGF inhibitor is administered prior to administration of the protein scaffolds described herein. In some embodiments, the VEGF inhibitor is administered following administration of the protein scaffolds described herein. In some embodiments, the VEGF inhibitor is administered during administration of the protein scaffolds described herein.

In some embodiments, the VEGF inhibitor is an antibody, an antigen binding fragment, a RNA interfering agent (RNAi), a small interfering RNA (siRNA), a short hairpin RNA (shRNA), a microRNA (miRNA), an antisense oligonucleotide, a peptide, a peptidomimetic, a small molecule, or an aptamer. In some embodiments, the VEGF inhibitor is pazopanib, bevacizumab, sunitinib, cabozantinib, sorafenib, axitinib, regorafenib, ponatinib, cabozantinib, vandetanib, ramucirumab, lenvatinib, aflibercept, or ziv-aflibercept.

Described herein, in some embodiments, are protein scaffolds for treating a disease or disorder resistant to VEGF treatment. In some embodiments, the disease or disorder is diabetes, obesity, cardiovascular disease, cancer, retinopathy, macular degeneration, or fibrosis.

Protein scaffolds described herein for treatment of a disease or disorder, in some embodiments, reduce expression of APJ-mediated angiogenesis. In some embodiments, the protein scaffolds described herein reduce APJ-mediated angiogenesis in a target cell. Methods for reducing APJ-mediated angiogenesis in a target cell, in some embodiments, comprise contacting the target cell with protein scaffolds as described herein for a time sufficient for binding of the protein scaffold to the APJ receptor, wherein the protein scaffold blocks interaction of the APJ receptor with a ligand of the APJ receptor. In some embodiments, the target cell is a cancer cell. In some embodiments, the cancer cell is from bladder cancer, bone cancer, brain cancer, breast cancer, colorectal cancer, eye cancer, head and neck cancer, kidney cancer, liver cancer, lung cancer, pancreatic cancer, prostate cancer, skin cancer, stomach cancer, thyroid cancer, or uterine cancer. In some embodiments, the target cell is a normal vascular cell.

### Pharmaceutically Acceptable Excipients, Carriers, and Diluents

In some embodiments, the protein scaffolds described herein are included in a pharmaceutical composition comprising one or more pharmaceutically acceptable excipients, carriers, and diluents. In some embodiments, the protein scaffolds described herein are administered suspended in a sterile solution. In some embodiments, the solution comprises 0.9% NaCl. In some embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, manno se, mannitol, sorbitol, sucrose, trehalose, and dextran 40; amino acids, for example, glycine or arginine; antioxidants, for example, ascorbic acid, methionine; or chelating agents, for example, EDTA or EGTA.

In some embodiments, the protein scaffolds described herein are shipped/stored lyophilized and reconstituted before administration. In some embodiments, lyophilized antibody formulations comprise a bulking agent such as, mannitol, sorbitol, sucrose, trehalose, dextran 40, or combinations thereof. In some embodiments, the lyophilized formulation is contained in a vial comprised of glass or other suitable non-reactive material. In some embodiments, the protein scaffolds when formulated, whether reconstituted or not, are buffered at a certain pH, generally less than 7.0. In some embodiments, the pH is between 4.5 and 6.5, 4.5 and 6.0, 4.5 and 5.5, 4.5 and 5.0, or 5.0 and 6.0.

Also described herein are kits comprising one or more of the protein scaffolds described herein in a suitable container and one or more additional component selected from: instructions for use; a diluent, an excipient, a carrier, and a device for administration.

In some embodiments, described herein is a method of preparing a cancer treatment comprising admixing one or more pharmaceutically acceptable excipients, carriers, or diluents and the protein scaffolds described herein. In some embodiments, described herein is a method of preparing a cancer treatment for storage or shipping comprising lyophilizing one or more the protein scaffolds described herein.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Generation of Protein Scaffolds

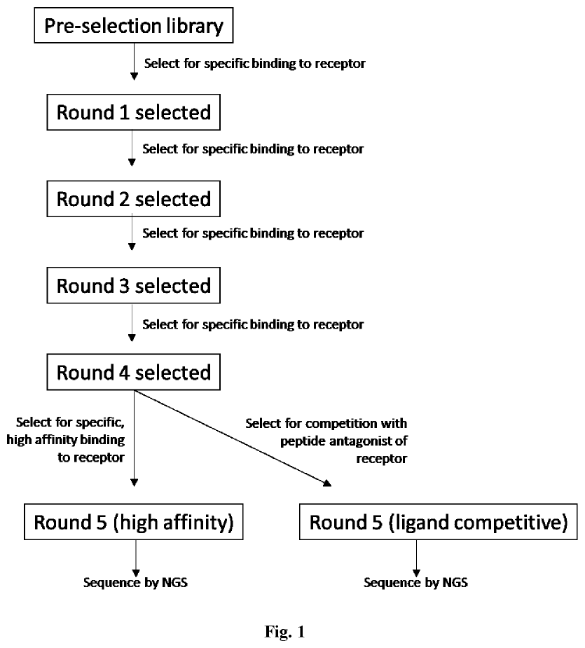

Protein scaffolds were generated according to a schema as seen in FIG. 1.

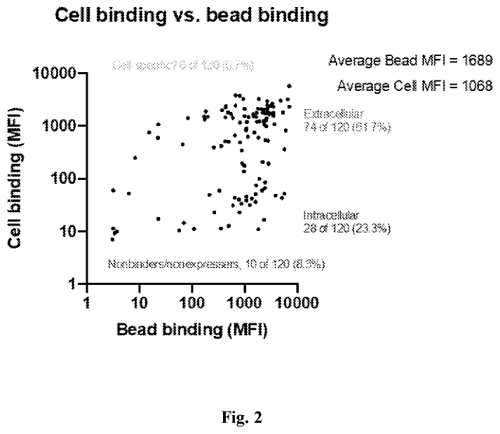

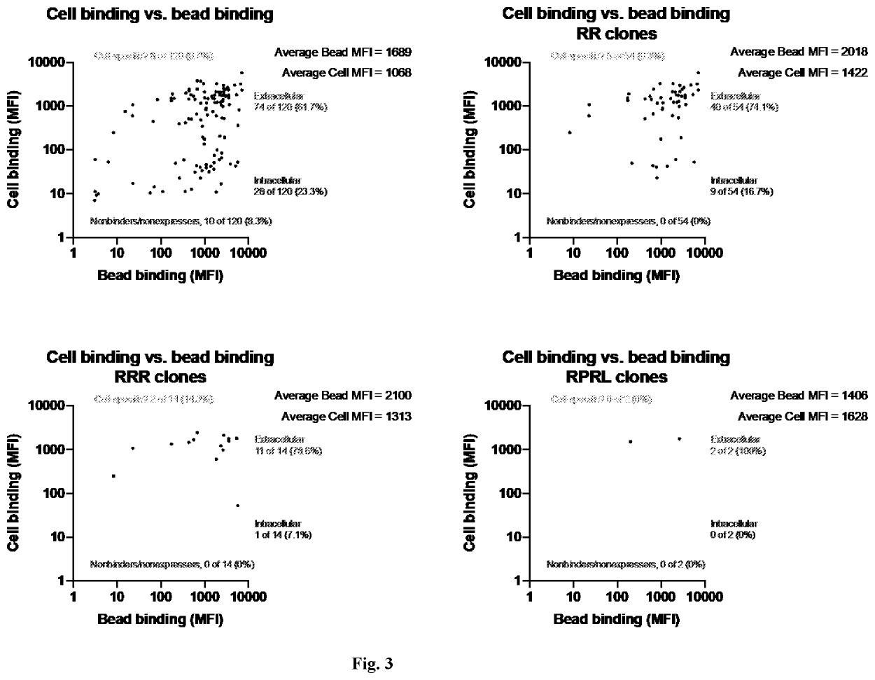

Following gene synthesis, 120 clones were transfected. The supernatant from these Fc fusion transfections were used to stain apelin (APJ)-expressing cells, and recombinant APJ on beads. As seen in FIG. 2, the majority of hits have detectable expression and clear binding to beads, cells, or both. About one-third of hits (28 clones) were intracellular (bead+, cell−) and about two-thirds of hits (74 clones) were extracellular (bead+, cell+). A population (8 clones) of cell-specific binders (bead−, cell+) was found to bind native conformations that are rarely presented by recombinant protein. As seen in FIG. 3, tandem arginines (“RR” and “RRR”) were highly enriched.

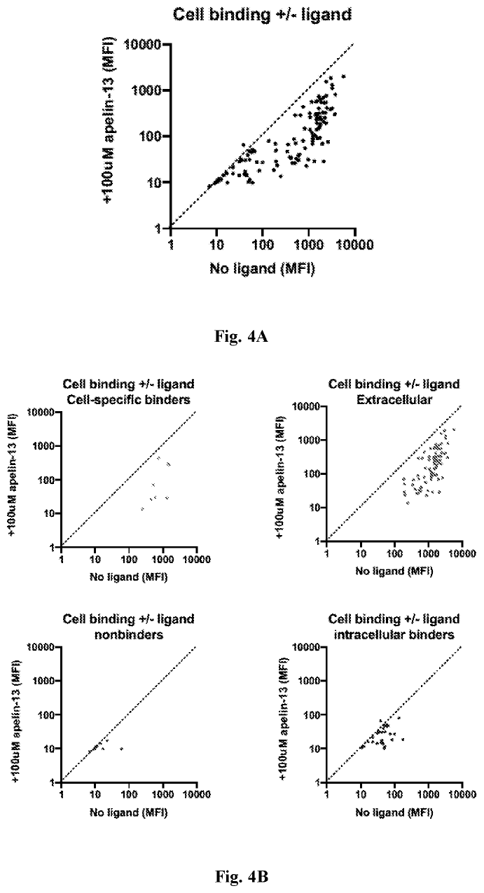

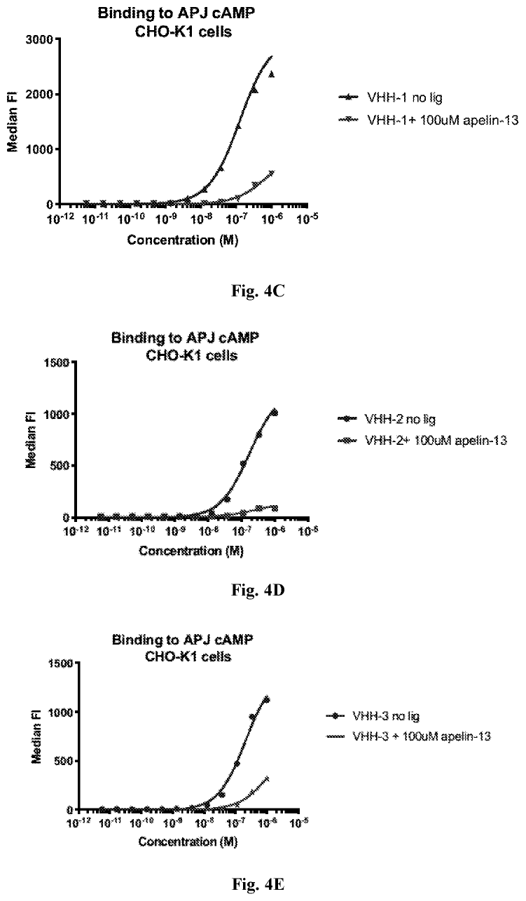

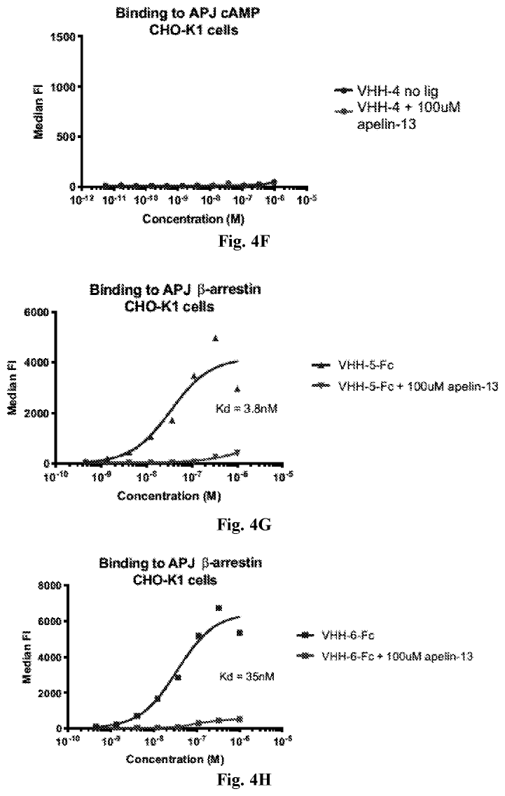

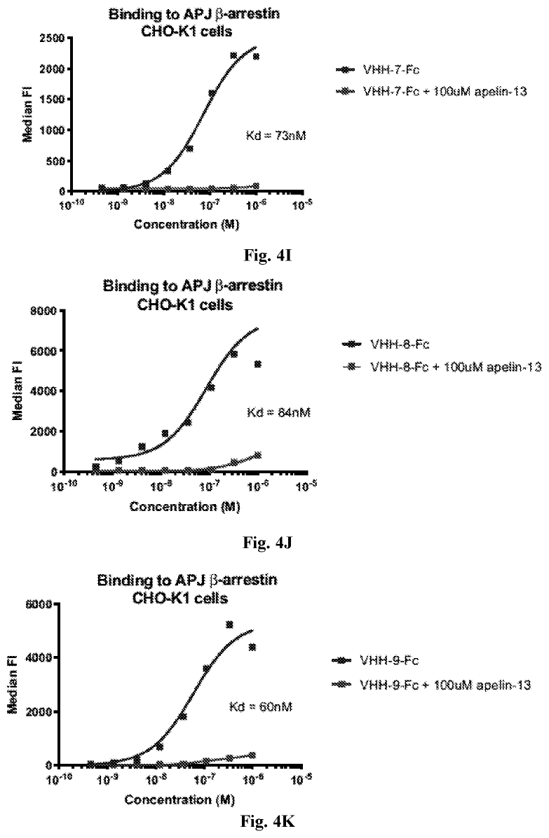

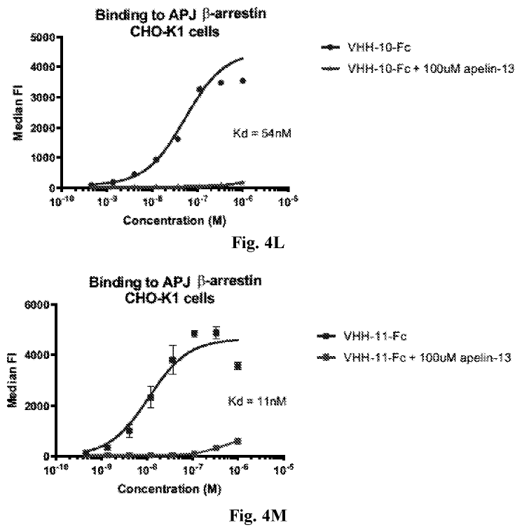

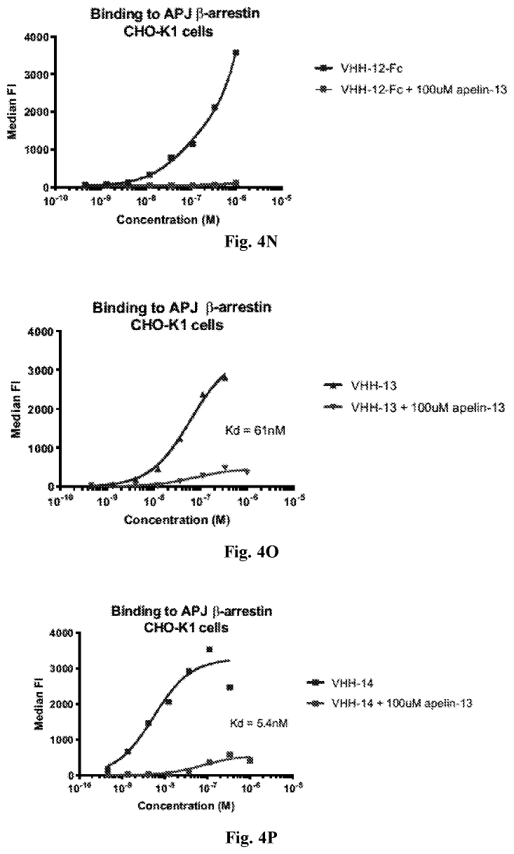

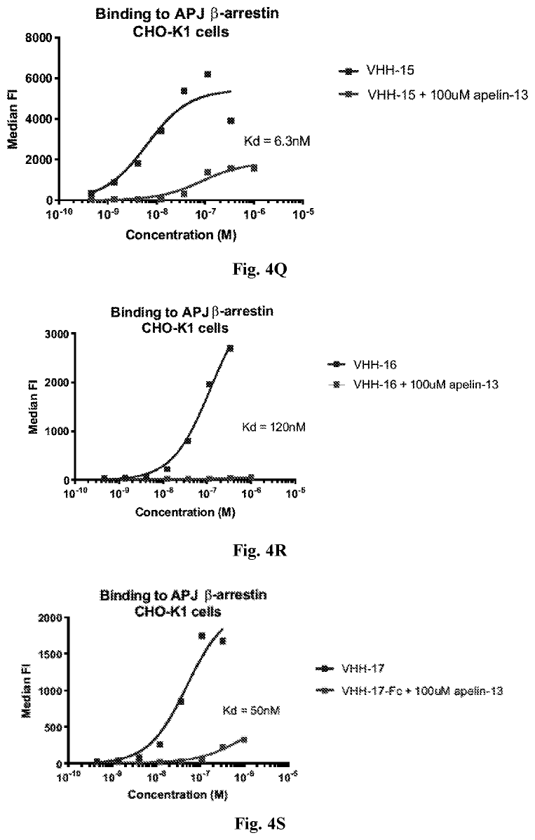

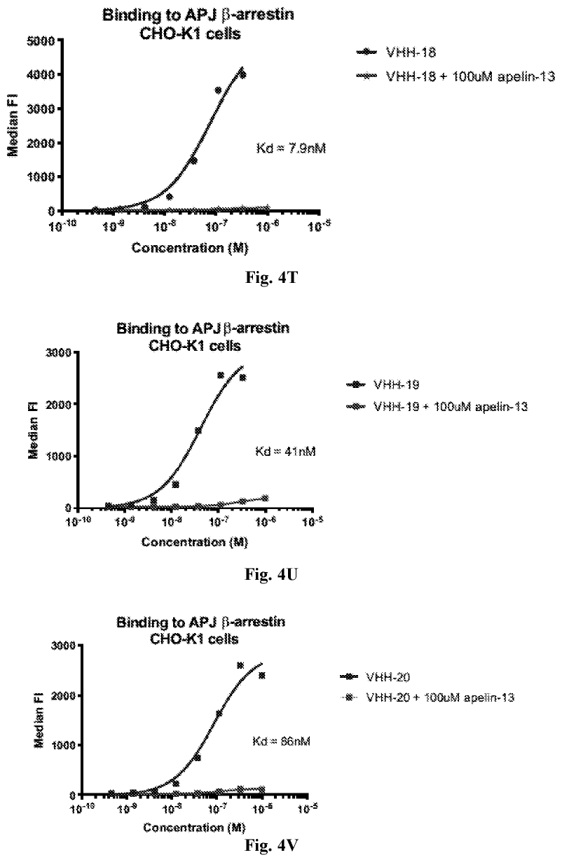

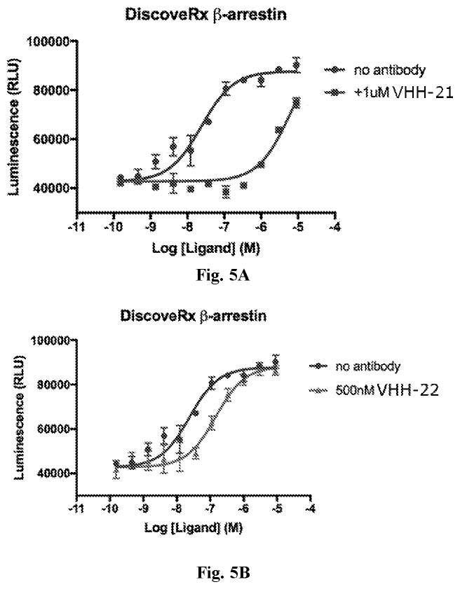

The clones were then assessed for binding and competition with native ligand. As seen in FIGS. 4A-4B, the clones that bound to extracellular APJ have reduced binding in competition with 100 uM APJ ligand. Based on the analysis, 82 clones were identified.

Twenty clones were then tested for specific binding to APJ-expressing cells. Nineteen of the twenty clones exhibited specific binding to APJ-expressing cells and all clones were strongly competed by apelin-13 (FIGS. 4C-4V). The Kd values ranged from about 3 nanomolar (nM) to about 200 nM, with a median of 54 nM.

### Example 2: APJ Negative Allosteric Modulators

Three APJ negative modulators were developed using similar methods described in Example 1.

FIGS. 5A-5C show graphs of the effects of three negative allosteric modulators on β-arrestin signaling. VHH-21 corresponds to SEQ ID NO: 67. VHH-22 corresponds to SEQ ID NO: 68, and VHH-23 corresponds to SEQ ID NO: 69. The Kd, Ki, and fold-suppression with VHH-21, VHH-22, and VHH-23, are shown in Table 5.

TABLE 5

| | | | Fold |
|---|---|---|---|
| | | | suppression |
| | Kd | Ki | (saturated) |
| VHH-21 | 61 nM | 3 nM | 247-fold |
| VHH-22 | 13 nM | Not | 5.9-fold |
| | | determined | |
| VHH-23 | Not | 39 nM | 8.5-fold |
| | determined | | |

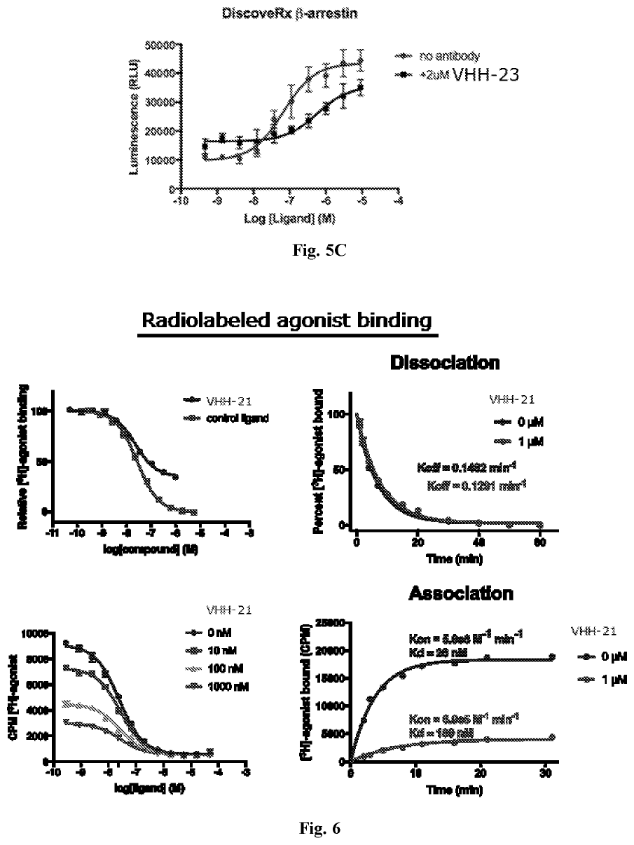

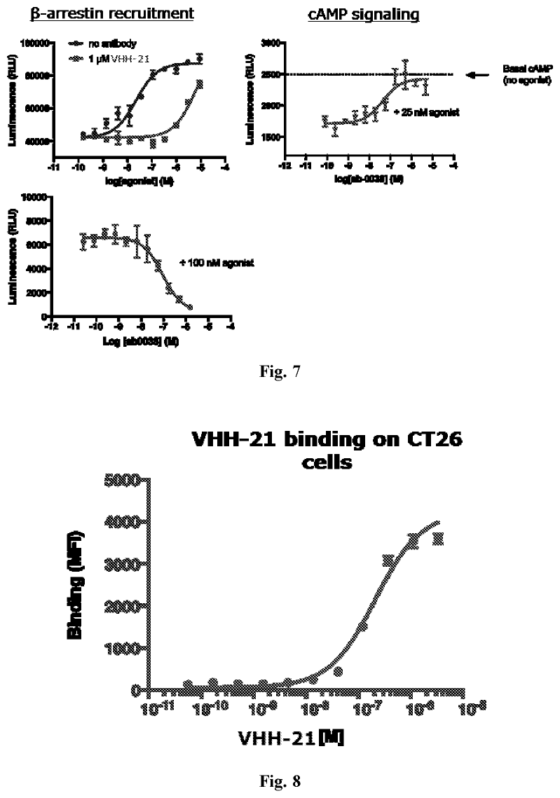

FIG. 6 shows data from radiolabeled agonist binding assays demonstrating that VHH-21 is an inhibitor of APJ-ligand binding. FIG. 7 shows effects of VHH-21 on cAMP and β-arrestin signaling. As seen in FIG. 7, VHH-21 blocks both cAMP and β-arrestin signaling through the APJ receptor and at a saturating concentration, VHH-21 induces a more than a 230-fold reduction in the EC50 of APJ ligand.

### Example 3: APJ Modulators in a Mouse Tumor Model

Negative allosteric modulators of APJ were used in a syngeneic mouse tumor model.

The CT26 mouse colon tumor line was used. Negative allosteric modulator VHH-21 was found to bind to mouse APJ receptor expressed on CT26 cells (FIG. 8) with an affinity of about 100 nM and to human cells about 30 nM.

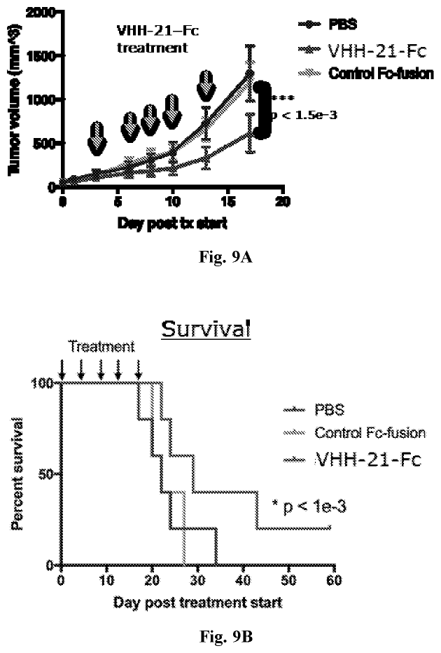

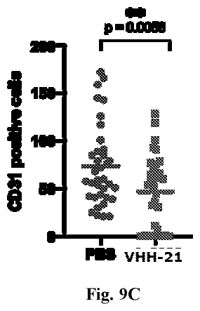

The effects of VHH-21 on tumor growth and survival were determined. Mice were engrafted with subcutaneous CT26 tumors, and treated with PBS, control Fc-fusion protein, or VHH-21-Fc by intraperitoneal injection 3 times per week. The Fc fusion proteins are the aglycosylated form of human IgG1, which has no effector function (i.e. no ADCC or ADCP). VHH-21-Fc significantly slowed the growth of tumors by 17 days post-engraftment (FIG. 9A) and significantly improved survival (FIG. 9B). VHH-21 also resulted in reduced angiogenesis in CT26 tumors. Tumors from VHH-21 or mock treated mice were imaged to quantify CD31⁺ cells. CD31 (PECAM-1) is expressed on endothelial cells and is a marker of angiogenesis. VHH-21 treated mouse tumors had significantly fewer CD31⁺ cells (FIG. 9C).

### Example 4: Nanobody to IgG Conversion

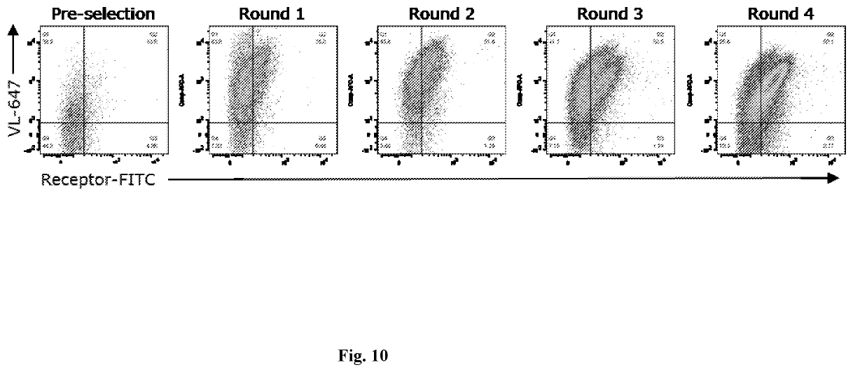

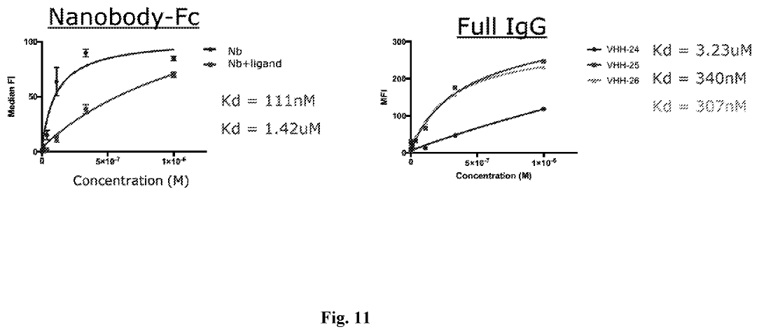

Nanobody candidates with high-affinity, specific binding to its target on cells, and a potent negative allosteric modulator were used for IgG conversion. A yeast display library in which CDR1, CDR2, and CDR3 from the original nanobody were ported into fully human VH scaffolds was generated. The library comprised sequence variation at CDR1 or CDR2 but not the CDR3 sequence. The library was subjected to multiple rounds of selection for VL pairing and receptor binding. As seen in FIG. 10, after 4 rounds of selection (with attendant clears), the library converged on a VL+, Receptor+ population. The clones from the 3^{rd} round of selection were further analyzed. As seen in FIG. 11, 2 out of 3 (VHH-25 and VHH-26) clones tested show saturable binding to target cells. The sequences for VHH-25 and VHH-26 are seen in Table 6.

TABLE 6

| | | SEQ | | SEQ |
|---|---|---|---|---|
| | | ID | | ID |
| | VH Sequence | NO: | VL Sequence | NO: |
| VHH-25 | QVQLLESGGGLVKPGGSLRLS | 93 | DIQMTQSPSSLSASVGDRVTITC | 95 |
| | CAASGFTFSDYYMSWIRQAPG | | RASQSISSYLNWYQQKPGKAPK | |
| | KGLEWVAGINYGSNTYYADS | | LLIYAASSLQSGVPSRFSGSGSG | |
| | VKGRFTISRDNAKNSLYLQMN | | TDFTLTISSLQPEDFATYYCQQS | |
| | SLRAEDTAVYYCAASQRRAY | | YSTPCQQSYTSPRTFGQGTKVEI | |
| | AARIDLFSYHTYGGQGTLVTV | | K | |
| | SS | | | |
| VHH-26 | QVQLLESGGGLVKPGGSLRLS | 94 | DIQMTQSPSSLSASVGDRVTITC | 96 |
| | CAASGFTFSDYYMSWIRQAPG | | RASQSISSYLNWYQQKPGKAPK | |
| | KGLEWVAGINYGSNTYYADS | | LLIYAASSLQSGVPSRFSGSGSG | |
| | VKGRFTISRDNAKNSLYLQMN | | TDFTLTISSLQPEDFATYYCQQS | |
| | SLRAEDTAVYYCAASQRRAY | | YSTPCQQTYTTPRTFGQGTKVEI | |
| | AARIDLFSYHTYWGQGTLVTV | | K | |
| | SS | | | |

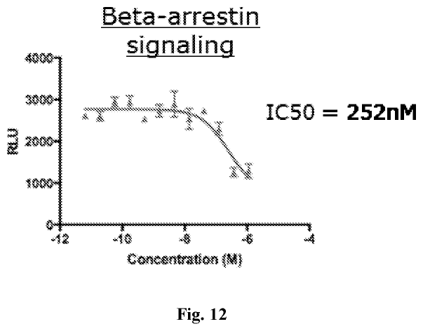

VHH-26 was further tested in β-arrestin signaling and was determined to be a functional, full IgG negative allosteric modulator of its target GPCR (FIG. 12).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A protein scaffold comprising an apelin (APJ) receptor binding domain, wherein the protein scaffold comprises a variable heavy chain (VH) region or VHH region, wherein the VH region or VHH region comprises:
a first sequence CAX₁X₂X₃X₄X₅X₆X₇X₈X₉X₁₀X₁₁X₁₂X₁₃X₁₄X₁₅X₁₆X₁₇X₁₈X₁₉X₂₀YW (SEQ ID NO: 89), wherein:
X₁ is selected from Y, G, S, D, T, R, A, L, V, P, N, F, E, I, W, Q, K, or H;
X₂ is selected from Y, G, S, D, T, R, A, L, V, P, N, F, E, I, W, Q, K, or H;
X₃ is selected from Y, G, S, D, T, R, A, L, V, P, N, F, E, I, W, Q, K, or H;
X₄ is selected from Y, G, S, D, T, R, A, L, V, P, N, F, E, I, W, Q, K, or H;
X₅ is selected from Y, G, S, D, T, R, A, L, V, P, N, F, E, I, W, Q, K, or H;
X₆ is selected from Y, G, S, D, T, R, A, L, V, P, N, F, E, I, W, Q, K, or H;
X₇ is selected from Y, G, S, D, T, R, A, L, V, P, N, F, E, I, W, Q, K, or H;
X₈ is either present or absent, if present, is selected from Y, G, S, D, T, R, A, L, V, P, N, F, E, I, W, Q, K, or H;
X₉ is either present or absent, if present, is selected from Y, G, S, D, T, R, A, L, V, P, N, F, E, I, W, Q, K, or H;
X₁₀ is either present or absent, if present, is selected from Y, G, S, D, T, R, A, L, V, P, N, F, E, I, W, Q, K, or H;
X₁₁ is either present or absent, if present, is selected from Y, G, S, D, T, R, A, L, V, P, N, F, E, I, W, Q, K, or H;
X₁₂ is either present or absent, if present, is selected from Y, G, S, D, T, R, A, L, V, P, N, F, E, I, W, Q, K, or H;
X₁₃ is either present or absent, if present, is selected from Y, G, S, D, T, R, A, L, V, P, N, F, E, I, W, Q, K, or H;
X₁₄ is either present or absent, if present, is selected from Y, G, S, D, T, R, A, L, V, P, N, F, E, I, W, Q, K, or H;
X₁₅ is either present or absent, if present, is selected from Y, G, S, D, T, R, A, L, V, P, N, F, E, I, W, Q, K, or H;
X₁₆ is either present or absent, if present, is selected from Y, G, S, D, T, R, A, L, V, P, N, F, E, I, W, Q, K, or H;
X₁₇ is either present or absent, if present, is selected from Y, G, S, D, T, R, A, L, V, P, N, F, E, I, W, Q, K, or H;
X₁₈ is either present or absent, if present, is selected from Y, G, S, D, T, R, A, L, V, P, N, F, E, I, W, Q, K, or H;
X₁₉ is selected from F, H, L, or Y; and
X₂₀ is selected from Y, G, S, D, T, R, A, L, V, P, N, F, E, I, W, Q, K, or H.

2. The protein scaffold of claim 1, wherein at least two, at least three, or at least four of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R.

3.-4. (canceled)

5. The protein scaffold of claim 1, wherein two, three, or four of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ is an R.

6.-7. (canceled)

8. The protein scaffold of claim 1, wherein at least two, at least three, or at least four of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's.

9.-10. (canceled)

11. The protein scaffold of claim 1, wherein two, three, or four of X₁, X₂, X₃, X₄, X₅, X₆, X₇, X₈, X₉, X₁₀, X₁₁, X₁₂, X₁₃, X₁₄, X₁₅, X₁₆, X₁₇, and X₁₈ are contiguous R's.

12.-13. (canceled)

14. The protein scaffold of claim 1, wherein:
each of X₄ and X₅ is independently an R;
each of X₆, X₇, and X₈ is independently an R;
each of X₂, X₃, and X₄ is independently an R;
each of X₅, X₆, and X₇ is independently an R;
each of X₇, X₈, X₁₄, and X₁₅ is independently an R;
each of X₅ and X₆ is independently an R;
each of X₄, X₅, X₁₁, and X₁₂ is independently an R;
each of X₁₄ and X₁₅ is independently an R;
each of X₃, X₄, and X₅ is independently an R; or
each of X₁₃ and X₁₄ is independently an R.

15.-23. (canceled)

24. The protein scaffold of claim 1, wherein the first sequence is a CDR3 sequence.

25. The protein scaffold of claim 1, wherein the first sequence is selected from SEQ ID NOs: 1-22.

26. The protein scaffold of claim 1, wherein the VH region further comprises:
a second sequence GX₂₁IX₂₂X₂₃X₂₄X₂₅X₂₆M (SEQ ID NO: 90), wherein:
X₂₁ is selected from Y, T, S, or N;
X₂₂ is selected from F or S;
X₂₃ is selected from Y, G, S, D, T, R, A, L, V, P, N, F, E, I, W, Q, K, or H;
X₂₄ is selected from Y, G, S, D, T, R, A, L, V, P, N, F, E, I, W, Q, K, or H;
X₂₅ is selected from Y, G, S, D, T, R, A, L, V, P, N, F, E, I, W, Q, K, or H; and
X₂₆ is selected from Y, G, S, D, T, R, A, L, V, P, N, F, E, I, W, Q, K, or H.

27. The protein scaffold of claim 26, wherein at least two, at least three, or at least four of X₂₃, X₂₄, X₂₅, and X₂₆ is an R.

28.-29. (canceled)

30. The protein scaffold of claim 26, wherein two, three, or four of X₂₃, X₂₄, X₂₅, and X₂₆ is an R.

31.-32. (canceled)

33. The protein scaffold of claim 26, wherein at least two, at least three, or at least four of X₂₃, X₂₄, X₂₅, and X₂₆ are contiguous R's.

34.-35. (canceled)

36. The protein scaffold of claim 26, wherein two, three, or four of X₂₃, X₂₄, X₂₅, and X₂₆ are contiguous R's.

37.-38. (canceled)

39. The protein scaffold of claim 26, wherein each of X₂₅ and X₂₆ is independently an R.

40. The protein scaffold of claim 26, wherein the second sequence is a CDR1 sequence.

41. The protein scaffold of claim 26, wherein the second sequence is selected from SEQ ID NOs: 23-44.

42. The protein scaffold of claim 1, wherein the VH region further comprises:
a third sequence EX₂₇VAX₂₈IX₂₉X₃₀GX₃₁X₃₂TX₃₃Y (SEQ ID NO: 91) or EX₃₄VAIX₃₅X₃₆GX₃₇X₃₇TX₃₉Y (SEQ ID NO: 92), wherein:
X₂₇ is selected from F or L;
X₂₈ is selected from A, G, S, or T;
X₂₉ is selected from A, D, G, N, S, or T;
X₃₀ is selected from Y, G, S, D, T, R, A, L, V, P, N, F, E, I, W, Q, K, or H;
X₃₁ is selected from A, G, S, or T;
X₃₂ is selected from I, N, S, T;
X₃₃ is selected from N or Y;
X₃₄ is selected from F or L;
X₃₅ is selected from A, G, S, or T;
X₃₆ is selected from Y, G, S, D, T, R, A, L, V, P, N, F, E, I, W, Q, K, or H;
X₃₇ is selected from A, G, S, or T;
X₃₈ is selected from I, N, S, T; and
X₃₉ is selected from N or Y.

43. The protein scaffold of claim 42, wherein the third sequence is a CDR2 sequence.

44. The protein scaffold of claim 42, wherein the third sequence is selected from SEQ ID NOs: 45-66.

45. The protein scaffold of claim 1, wherein the protein scaffold blocks APJ receptor ligand binding.

46. The protein scaffold of claim 1, wherein the protein scaffold is an allosteric modulator of the APJ receptor.

47. The protein scaffold of claim 46, wherein the protein scaffold is a negative allosteric modulator of the APJ receptor.

48. The protein scaffold of claim 1, wherein the protein scaffold is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, a nanobody, or ab antigen-binding fragments thereof.

49. The protein scaffold of claim 1, wherein the protein scaffold is a nano body.

50. The protein scaffold of claim 1, comprising a sequence selected from SEQ ID NOs: 67-88.

51. The protein scaffold of claim 1, wherein the protein scaffold comprises at least a 40%, 50%, 60%, 70%, 80%, or 90% inhibition of APJ signaling at a concentration range of about 1 nM to about 100 nM.

52. The protein scaffold of claim 1, wherein the protein scaffold comprises at least a 2-fold, 5-fold, 10-fold, 50-fold, 100-fold, 150-fold, 200-fold, or 250-fold inhibition of APJ signaling at a concentration range of about 1 nM (nanomolar) to about 100 nM.

53. The protein scaffold of claim 1, wherein the protein scaffold comprises a Kd less than 200 nM, less than 150 nM, less than 100 nM, less than 75 nM, less than 50 nM, less than 25 nM, or less than 10 nM.

54. A pharmaceutical composition comprising a protein scaffold of claim 1, and a pharmaceutically acceptable excipient.

55. The pharmaceutical composition of claim 54, wherein the pharmaceutical composition is formulated for systemic administration or parenteral administration.

56. (canceled)

57. An isolated nucleic acid molecule encoding the protein scaffold of claim 1.

58. A vector comprising a nucleic acid sequence encoding the protein scaffold of claim 1.

59. The vector of claim 58, wherein the vector is a viral vector.

60. The vector of claim 59, wherein the viral vector comprises a retrovirus, an adenovirus, an adeno associated virus, a lentivirus, or a herpes virus.

61. A host cell producing a protein scaffold of claim 1.

62. A method of treating a disease or disorder characterized by aberrant APJ signaling in a subject in need thereof, comprising administering to the subject the protein scaffold, or a nucleic acid that encodes for the protein scaffold, of claim 1.

63. The method of claim 62, wherein the disease or disorder is diabetes, obesity, cardiovascular disease, retinopathy, macular degeneration, fibrosis, cancer, bladder cancer, brain cancer, breast cancer, bladder cancer, bone cancer, cervical cancer, colorectal cancer, esophageal cancer, head and neck cancer, kidney cancer, liver cancer, lung cancer, ovarian cancer, pancreatic cancer, prostate cancer, skin cancer, stomach cancer, thyroid cancer, or uterine cancer.

64.-65. (canceled)

66. The method of claim 62, wherein the protein scaffold is administered subcutaneously, intraperitoneally, intravenously, intramuscularly, or intratumorally.

67. The method of claim 62, further comprising administer a vascular endothelial growth factor (VEGF) inhibitor.

68. The method of claim 67, wherein the VEGF inhibitor is an antibody, an antigen binding fragment, a RNA interfering agent (RNAi), a small interfering RNA (siRNA), a short hairpin RNA (shRNA), a microRNA (miRNA), an antisense oligonucleotide, a peptide, a peptidomimetic, a small molecule, or an aptamer.

69. The method of claim 67, wherein the VEGF inhibitor is pazopanib, bevacizumab, sunitinib, cabozantinib, sorafenib, axitinib, regorafenib, ponatinib, vandetanib, ramucirumab, lenvatinib, aflibercept, or ziv-aflibercept.

70. The method of claim 62, wherein the subject is resistant to VEGF treatment.

71.-85. (canceled)

86. A method of reducing APJ-mediated angiogenesis in a target cell, comprising:
contacting the target cell with a protein scaffold of claim 1 for a time sufficient for binding of the protein scaffold to the APJ receptor, wherein the protein scaffold blocks interaction of the APJ receptor with a ligand of the APJ receptor.

87. The method of claim 86, wherein the target cell is a normal vascular cell or a cancer cell.

88. The method of claim 87, wherein the cancer cell is from bladder cancer, bone cancer, brain cancer, breast cancer, colorectal cancer, eye cancer, head and neck cancer, kidney cancer, liver cancer, lung cancer, pancreatic cancer, prostate cancer, skin cancer, stomach cancer, thyroid cancer, or uterine cancer.

89. (canceled)


SEQUENCE LISTING (96 sequences)

SEQ ID NO: 1 - protein, 22 aa, Artificial Sequence
  Description of Artificial Sequence Synthetic peptide
CAASQRRAYAARIDLFSYHTYW

SEQ ID NO: 2 - protein, 22 aa, Artificial Sequence
  Description of Artificial Sequence Synthetic peptide
CAARLSERRRLEGYWSAVHVYW

SEQ ID NO: 3 - protein, 21 aa, Artificial Sequence
  Description of Artificial Sequence Synthetic peptide
CAASCRAYASRIDLFSYHTYW

SEQ ID NO: 4 - protein, 20 aa, Artificial Sequence
  Description of Artificial Sequence Synthetic peptide
CAAYRKSYTLDTGKRAYTYW

SEQ ID NO: 5 - protein, 18 aa, Artificial Sequence
  Description of Artificial Sequence Synthetic peptide
CAVRRRYFLDPNTRHVYW

SEQ ID NO: 6 - protein, 23 aa, Artificial Sequence
  Description of Artificial Sequence Synthetic peptide
CAANRQLRGLYLERYRYRTYLYW

SEQ ID NO: 7 - protein, 20 aa, Artificial Sequence
  Description of Artificial Sequence Synthetic peptide
CAVRGGRRRTLQGGSYHLYW

SEQ ID NO: 8 - protein, 17 aa, Artificial Sequence
  Description of Artificial Sequence Synthetic peptide
CAVYIADLYVRSYHSYW

SEQ ID NO: 9 - protein, 20 aa, Artificial Sequence
  Description of Artificial Sequence Synthetic peptide
CAVYAQRLTYIASARYHRYW

SEQ ID NO: 10 - protein, 22 aa, Artificial Sequence
  Description of Artificial Sequence Synthetic peptide
CAVRSPFVRRLDELGRRYHVYW

SEQ ID NO: 11 - protein, 22 aa, Artificial Sequence
  Description of Artificial Sequence Synthetic peptide
CAVQYGRRSSLDGGRYNQHTYW

SEQ ID NO: 12 - protein, 20 aa, Artificial Sequence
  Description of Artificial Sequence Synthetic peptide
CAVYGRRISIESRRGVFSYW

SEQ ID NO: 13 - protein, 21 aa, Artificial Sequence
  Description of Artificial Sequence Synthetic peptide
CAVSYRGYARGELGGRRYQYW

SEQ ID NO: 14 - protein, 20 aa, Artificial Sequence
  Description of Artificial Sequence Synthetic peptide
CAARGRAYSVDVAVAYHTYW

SEQ ID NO: 15 - protein, 20 aa, Artificial Sequence
  Description of Artificial Sequence Synthetic peptide
CAAWRRRIDYHVRVSRLNYW

SEQ ID NO: 16 - protein, 20 aa, Artificial Sequence
  Description of Artificial Sequence Synthetic peptide
CAARYVPYTRQIRYARYVYW

SEQ ID NO: 17 - protein, 20 aa, Artificial Sequence
  Description of Artificial Sequence Synthetic peptide
CAADKKRRRSLLNYLHFRYW

SEQ ID NO: 18 - protein, 22 aa, Artificial Sequence
  Description of Artificial Sequence Synthetic peptide
CAVVGRYRSYDITSYRSTYHYW

SEQ ID NO: 19 - protein, 20 aa, Artificial Sequence
  Description of Artificial Sequence Synthetic peptide
CAVNVLRFQPPDGARKLRYW

SEQ ID NO: 20 - protein, 24 aa, Artificial Sequence
  Description of Artificial Sequence Synthetic peptide
CAVPDGYRPHLEPDRRGYSAYIYW

SEQ ID NO: 21 - protein, 16 aa, Artificial Sequence
  Description of Artificial Sequence Synthetic peptide
CAVRGGIESIYSFRYW

SEQ ID NO: 22 - protein, 20 aa, Artificial Sequence
  Description of Artificial Sequence Synthetic peptide
CAATTQRSKHGLRLRALLYW

SEQ ID NO: 23 - protein, 9 aa, Artificial Sequence
  Description of Artificial Sequence Synthetic peptide
GSISVGYLM

SEQ ID NO: 24 - protein, 9 aa, Artificial Sequence
  Description of Artificial Sequence Synthetic peptide
GNIFRGRVM

SEQ ID NO: 25 - protein, 9 aa, Artificial Sequence
  Description of Artificial Sequence Synthetic peptide
GNISPFRAM

SEQ ID NO: 26 - protein, 9 aa, Artificial Sequence
  Description of Artificial Sequence Synthetic peptide
GTIFHRGYM

SEQ ID NO: 27 - protein, 9 aa, Artificial Sequence
  Description of Artificial Sequence Synthetic peptide
GTISSYVYM

SEQ ID NO: 28 - protein, 8 aa, Artificial Sequence
  Description of Artificial Sequence Synthetic peptide
GSISDYFM

SEQ ID NO: 29 - protein, 9 aa, Artificial Sequence
  Description of Artificial Sequence Synthetic peptide
GTISTLRTM

SEQ ID NO: 30 - protein, 9 aa, Artificial Sequence
  Description of Artificial Sequence Synthetic peptide
GTISLHRYM

SEQ ID NO: 31 - protein, 9 aa, Artificial Sequence
  Description of Artificial Sequence Synthetic peptide
GSIFYSRTM

SEQ ID NO: 32 - protein, 9 aa, Artificial Sequence
  Description of Artificial Sequence Synthetic peptide
GYISYTSYM

SEQ ID NO: 33 - protein, 9 aa, Artificial Sequence
  Description of Artificial Sequence Synthetic peptide
GSIFSTWRM

SEQ ID NO: 34 - protein, 9 aa, Artificial Sequence
  Description of Artificial Sequence Synthetic peptide
GYISNYYYM

SEQ ID NO: 35 - protein, 9 aa, Artificial Sequence
  Description of Artificial Sequence Synthetic peptide
GSISYNAYM

SEQ ID NO: 36 - protein, 9 aa, Artificial Sequence
  Description of Artificial Sequence Synthetic peptide
GTIFGYRYM

SEQ ID NO: 37 - protein, 8 aa, Artificial Sequence
  Description of Artificial Sequence Synthetic peptide
GTISYWTM

SEQ ID NO: 38 - protein, 9 aa, Artificial Sequence
  Description of Artificial Sequence Synthetic peptide
GSIFAKATM

SEQ ID NO: 39 - protein, 9 aa, Artificial Sequence
  Description of Artificial Sequence Synthetic peptide
GYISAVRTM

SEQ ID NO: 40 - protein, 9 aa, Artificial Sequence
  Description of Artificial Sequence Synthetic peptide
GTISRSAYM

SEQ ID NO: 41 - protein, 9 aa, Artificial Sequence
  Description of Artificial Sequence Synthetic peptide
GTISAAAYM

SEQ ID NO: 42 - protein, 9 aa, Artificial Sequence
  Description of Artificial Sequence Synthetic peptide
GSIFRYRRM

SEQ ID NO: 43 - protein, 9 aa, Artificial Sequence
  Description of Artificial Sequence Synthetic peptide
GYISPYRYM

SEQ ID NO: 44 - protein, 9 aa, Artificial Sequence
  Description of Artificial Sequence Synthetic peptide
GTISYTTRM

SEQ ID NO: 45 - protein, 14 aa, Artificial Sequence
  Description of Artificial Sequence Synthetic peptide
ELVAGINYGSNTYY

SEQ ID NO: 46 - protein, 14 aa, Artificial Sequence
  Description of Artificial Sequence Synthetic peptide
EFVAGIDYGASTNY

SEQ ID NO: 47 - protein, 14 aa, Artificial Sequence
  Description of Artificial Sequence Synthetic peptide
EFVAAINTGAITNY

SEQ ID NO: 48 - protein, 14 aa, Artificial Sequence
  Description of Artificial Sequence Synthetic peptide
EFVATITSGSSTNY

SEQ ID NO: 49 - protein, 14 aa, Artificial Sequence
  Description of Artificial Sequence Synthetic peptide
ELVAAINRGGTTNY

SEQ ID NO: 50 - protein, 14 aa, Artificial Sequence
  Description of Artificial Sequence Synthetic peptide
EFVAGITAGGITNY

SEQ ID NO: 51 - protein, 14 aa, Artificial Sequence
  Description of Artificial Sequence Synthetic peptide
EFVASIGNGGSTYY

SEQ ID NO: 52 - protein, 14 aa, Artificial Sequence
  Description of Artificial Sequence Synthetic peptide
ELVASINRGGSTYY

SEQ ID NO: 53 - protein, 14 aa, Artificial Sequence
  Description of Artificial Sequence Synthetic peptide
EFVATIGLGGITNY

SEQ ID NO: 54 - protein, 14 aa, Artificial Sequence
  Description of Artificial Sequence Synthetic peptide
ELVATINQGAITNY

SEQ ID NO: 55 - protein, 14 aa, Artificial Sequence
  Description of Artificial Sequence Synthetic peptide
EFVAGIDRGGTTYY

SEQ ID NO: 56 - protein, 14 aa, Artificial Sequence
  Description of Artificial Sequence Synthetic peptide
ELVATIGSGGNTYY

SEQ ID NO: 57 - protein, 14 aa, Artificial Sequence
  Description of Artificial Sequence Synthetic peptide
EFVASITYGGNTYY

SEQ ID NO: 58 - protein, 14 aa, Artificial Sequence
  Description of Artificial Sequence Synthetic peptide
EFVASIGRGTSTNY

SEQ ID NO: 59 - protein, 14 aa, Artificial Sequence
  Description of Artificial Sequence Synthetic peptide
ELVASISGGGITYY

SEQ ID NO: 60 - protein, 14 aa, Artificial Sequence
  Description of Artificial Sequence Synthetic peptide
ELVATITTGTTTNY

SEQ ID NO: 61 - protein, 14 aa, Artificial Sequence
  Description of Artificial Sequence Synthetic peptide
EFVAAISYGSITNY

SEQ ID NO: 62 - protein, 14 aa, Artificial Sequence
  Description of Artificial Sequence Synthetic peptide
EFVATIAGGGSTNY

SEQ ID NO: 63 - protein, 14 aa, Artificial Sequence
  Description of Artificial Sequence Synthetic peptide
EFVAGIDRGATTYY

SEQ ID NO: 64 - protein, 14 aa, Artificial Sequence
  Description of Artificial Sequence Synthetic peptide
EFVASISPGTNTNY

SEQ ID NO: 65 - protein, 13 aa, Artificial Sequence
  Description of Artificial Sequence Synthetic peptide
ELVAIGRGGNTYY

SEQ ID NO: 66 - protein, 14 aa, Artificial Sequence
  Description of Artificial Sequence Synthetic peptide
EFVAAIAGGGSTNY

SEQ ID NO: 67 - protein, 126 aa, Artificial Sequence
  Description of Artificial Sequence Synthetic polypeptide
QVQLQESGGGLVQAGGSLRLSCAASGSISVGYLMGWYRQAPGKERELVAGINYGSNTYYADSVKGRFTISRDNAKNTVYLQMNSLKPEDTAVYYCAASQRRAYAARIDLFSYHTYWGQGTQVTVSS

SEQ ID NO: 68 - protein, 126 aa, Artificial Sequence
  Description of Artificial Sequence Synthetic polypeptide
QVQLQESGGGLVQAGGSLRLSCAASGNIFRGRVMGWYRQAPGKEREFVAGIDYGASTNYADSVKGRFTISRDNAKNTVYLQMNSLKPEDTAVYYCAARLSERRRLEGYWSAVHVYWGQGTQVTVSS

SEQ ID NO: 69 - protein, 125 aa, Artificial Sequence
  Description of Artificial Sequence Synthetic polypeptide
QVQLQESGGGLVQAGGSLRLSCAASGNISPFRAMGWYRQAPGKEREFVAAINTGAITNYADSVKGRFTISRDNAKNTVYLQMNSLKPEDTAVYYCAASCRAYASRIDLFSYHTYWGQGTQVTVSS

SEQ ID NO: 70 - protein, 127 aa, Artificial Sequence
  Description of Artificial Sequence Synthetic polypeptide
QVQLQESGGGLVQAGGSLRLSCAASGTIFHRGYMGWYRQAPGKEREFVATITSGSSTNYADSVKGRFTISRDNAKNTVYLQMNSLKPEDTAVYYCAAYRKSYTLDTGKRAYTYWGQGTQVTVSSGSG

SEQ ID NO: 71 - protein, 125 aa, Artificial Sequence
  Description of Artificial Sequence Synthetic polypeptide
QVQLQESGGGLVQAGGSLRLSCAASGTISSYVYMGWYRQAPGKERELVAAINRGGTTNYADSVKGRFTISRDNAKNTVYLQMNSLKPEDTAVYYCAVRRRYFLDPNTRHVYWGQGTQVTVSSGSG

SEQ ID NO: 72 - protein, 129 aa, Artificial Sequence
  Description of Artificial Sequence Synthetic polypeptide
QVQLQESGGGLVQAGGSLRLSCAASGSISDYFMGWYRQAPGKEREFVAGITAGGITNYADSVKGRFTISRDNAKNTVYLQMNSLKPEDTAVYYCAANRQLRGLYLERYRYRTYLYWGQGTQVTVSSGSG

SEQ ID NO: 73 - protein, 127 aa, Artificial Sequence
  Description of Artificial Sequence Synthetic polypeptide
QVQLQESGGGLVQAGGSLRLSCAASGTISTLRTMGWYRQAPGKEREFVASIGNGGSTYYADSVKGRFTISRDNAKNTVYLQMNSLKPEDTAVYYCAVRGGRRRTLQGGSYHLYWGQGTQVTVSSGSG

SEQ ID NO: 74 - protein, 124 aa, Artificial Sequence
  Description of Artificial Sequence Synthetic polypeptide
QVQLQESGGGLVQAGGSLRLSCAASGTISLHRYMGWYRQAPGKERELVASINRGGSTYYADSVKGRFTISRDNAKNTVYLQMNSLKPEDTAVYYCAVYIADLYVRSYHSYWGQGTQVTVSSGSG

SEQ ID NO: 75 - protein, 127 aa, Artificial Sequence
  Description of Artificial Sequence Synthetic polypeptide
QVQLQESGGGLVQAGGSLRLSCAASGSIFYSRTMGWYRQAPGKEREFVATIGLGGITNYADSVKGRFTISRDNAKNTVYLQMNSLKPEDTAVYYCAVYAQRLTYIASARYHRYWGQGTQVTVSSGSG

SEQ ID NO: 76 - protein, 129 aa, Artificial Sequence
  Description of Artificial Sequence Synthetic polypeptide
QVQLQESGGGLVQAGGSLRLSCAASGYISYTSYMGWYRQAPGKERELVATINQGAITNYADSVKGRFTISRDNAKNTVYLQMNSLKPEDTAVYYCAVRSPFVRRLDELGRRYHVYWGQGTQVTVSSGSG

SEQ ID NO: 77 - protein, 129 aa, Artificial Sequence
  Description of Artificial Sequence Synthetic polypeptide
QVQLQESGGGLVQAGGSLRLSCAASGSIFSTWRMGWYRQAPGKEREFVAGIDRGGTTYYADSVKGRFTISRDNAKNTVYLQMNSLKPEDTAVYYCAVQYGRRSSLDGGRYNQHTYWGQGTQVTVSSGSG

SEQ ID NO: 78 - protein, 127 aa, Artificial Sequence
  Description of Artificial Sequence Synthetic polypeptide
QVQLQESGGGLVQAGGSLRLSCAASGYISNYYYMGWYRQAPGKERELVATIGSGGNTYYADSVKGRFTISRDNAKNTVYLQMNSLKPEDTAVYYCAVYGRRISIESRRGVFSYWGQGTQVTVSSGSG

SEQ ID NO: 79 - protein, 128 aa, Artificial Sequence
  Description of Artificial Sequence Synthetic polypeptide
QVQLQESGGGLVQAGGSLRLSCAASGSISYNAYMGWYRQAPGKEREFVASITYGGNTYYADSVKGRFTISRDNAKNTVYLQMNSLKPEDTAVYYCAVSYRGYARGELGGRRYQYWGQGTQVTVSSGSG

SEQ ID NO: 80 - protein, 127 aa, Artificial Sequence
  Description of Artificial Sequence Synthetic polypeptide
QVQLQESGGGLVQAGGSLRLSCAASGTIFGYRYMGWYRQAPGKEREFVASIGRGTSTNYADSVKGRFTISRDNAKNTVYLQMNSLKPEDTAVYYCAARGRAYSVDVAVAYHTYWGQGTQVTVSSGSG

SEQ ID NO: 81 - protein, 126 aa, Artificial Sequence
  Description of Artificial Sequence Synthetic polypeptide
QVQLQESGGGLVQAGGSLRLSCAASGTISYWTMGWYRQAPGKERELVASISGGGITYYADSVKGRFTISRDNAKNTVYLQMNSLKPEDTAVYYCAAWRRRIDYHVRVSRLNYWGQGTQVTVSSGSG

SEQ ID NO: 82 - protein, 127 aa, Artificial Sequence
  Description of Artificial Sequence Synthetic polypeptide
QVQLQESGGGLVQAGGSLRLSCAASGSIFAKATMGWYRQAPGKERELVATITTGTTTNYADSVKGRFTISRDNAKNTVYLQMNSLKPEDTAVYYCAARYVPYTRQIRYARYVYWGQGTQVTVSSGSG

SEQ ID NO: 83 - protein, 127 aa, Artificial Sequence
  Description of Artificial Sequence Synthetic polypeptide
QVQLQESGGGLVQAGGSLRLSCAASGYISAVRTMGWYRQAPGKEREFVAAISYGSITNYADSVKGRFTISRDNAKNTVYLQMNSLKPEDTAVYYCAADKKRRRSLLNYLHFRYWGQGTQVTVSSGSG

SEQ ID NO: 84 - protein, 129 aa, Artificial Sequence
  Description of Artificial Sequence Synthetic polypeptide
QVQLQESGGGLVQAGGSLRLSCAASGTISRSAYMGWYRQAPGKEREFVATIAGGGSTNYADSVKGRFTISRDNAKNTVYLQMNSLKPEDTAVYYCAVVGRYRSYDITSYRSTYHYWGQGTQVTVSSGSG

SEQ ID NO: 85 - protein, 127 aa, Artificial Sequence
  Description of Artificial Sequence Synthetic polypeptide
QVQLQESGGGLVQAGGSLRLSCAASGTISAAAYMGWYRQAPGKEREFVAGIDRGATTYYADSVKGRFTISRDNAKNTVYLQMNSLKPEDTAVYYCAVNVLRFQPPDGARKLRYWGQGTQVTVSSGSG

SEQ ID NO: 86 - protein, 131 aa, Artificial Sequence
  Description of Artificial Sequence Synthetic polypeptide
QVQLQESGGGLVQAGGSLRLSCAASGSIFRYRRMGWYRQAPGKEREFVASISPGTNTNYADSVKGRFTISRDNAKNTVYLQMNSLKPEDTAVYYCAVPDGYRPHLEPDRRGYSAYIYWGQGTQVTVSSGSG

SEQ ID NO: 87 - protein, 122 aa, Artificial Sequence
  Description of Artificial Sequence Synthetic polypeptide
QVQLQESGGGLVQAGGSLRLSCAASGYISPYRYMGWYRQAPGKERELVAIGRGGNTYYADSVKGRFTISRDNAKNTVYLQMNSLKPEDTAVYYCAVRGGIESIYSFRYWGQGTQVTVSSGSG

SEQ ID NO: 88 - protein, 127 aa, Artificial Sequence
  Description of Artificial Sequence Synthetic polypeptide
QVQLQESGGGLVQAGGSLRLSCAASGTISYTTRMGWYRQAPGKEREFVAAIAGGGSTNYADSVKGRFTISRDNAKNTVYLQMNSLKPEDTAVYYCAATTQRSKHGLRLRALLYWGQGTQVTVSSGSG

SEQ ID NO: 89 - protein, 24 aa, Artificial Sequence
  Description of Artificial Sequence Synthetic peptide
  MOD_RES (3)..(3): Y, G, S, D, T, R, A, L, V, P, N, F, E, I, W, Q, K, or H
  MOD_RES (4)..(4): Y, G, S, D, T, R, A, L, V, P, N, F, E, I, W, Q, K, or H
  MOD_RES (5)..(5): Y, G, S, D, T, R, A, L, V, P, N, F, E, I, W, Q, K, or H
  MOD_RES (6)..(6): Y, G, S, D, T, R, A, L, V, P, N, F, E, I, W, Q, K, or H
  MOD_RES (7)..(7): Y, G, S, D, T, R, A, L, V, P, N, F, E, I, W, Q, K, or H
  MOD_RES (8)..(8): Y, G, S, D, T, R, A, L, V, P, N, F, E, I, W, Q, K, or H
  MOD_RES (9)..(9): Y, G, S, D, T, R, A, L, V, P, N, F, E, I, W, Q, K, or H
  MOD_RES (10)..(10): Y, G, S, D, T, R, A, L, V, P, N, F, E, I, W, Q, K, H, or absent
  MOD_RES (11)..(11): Y, G, S, D, T, R, A, L, V, P, N, F, E, I, W, Q, K, H, or absent
  MOD_RES (12)..(12): Y, G, S, D, T, R, A, L, V, P, N, F, E, I, W, Q, K, H, or absent
  MOD_RES (13)..(13): Y, G, S, D, T, R, A, L, V, P, N, F, E, I, W, Q, K, H, or absent
  MOD_RES (14)..(14): Y, G, S, D, T, R, A, L, V, P, N, F, E, I, W, Q, K, H, or absent
  MOD_RES (15)..(15): Y, G, S, D, T, R, A, L, V, P, N, F, E, I, W, Q, K, H, or absent
  MOD_RES (16)..(16): Y, G, S, D, T, R, A, L, V, P, N, F, E, I, W, Q, K, H, or absent
  MOD_RES (17)..(17): Y, G, S, D, T, R, A, L, V, P, N, F, E, I, W, Q, K, H, or absent
  MOD_RES (18)..(18): Y, G, S, D, T, R, A, L, V, P, N, F, E, I, W, Q, K, H, or absent
  MOD_RES (19)..(19): Y, G, S, D, T, R, A, L, V, P, N, F, E, I, W, Q, K, H, or absent
  MOD_RES (20)..(20): Y, G, S, D, T, R, A, L, V, P, N, F, E, I, W, Q, K, H, or absent
  MOD_RES (21)..(21): F, H, L, or Y
  MOD_RES (22)..(22): Y, G, S, D, T, R, A, L, V, P, N, F, E, I, W, Q, K, or H
CAXXXXXXXXXXXXXXXXXXXXYW

SEQ ID NO: 90 - protein, 9 aa, Artificial Sequence
  Description of Artificial Sequence Synthetic peptide
  MOD_RES (2)..(2): Y, T, S, or N
  MOD_RES (4)..(4): F or S
  MOD_RES (5)..(5): Y, G, S, D, T, R, A, L, V, P, N, F, E, I, W, Q, K, or H
  MOD_RES (6)..(6): Y, G, S, D, T, R, A, L, V, P, N, F, E, I, W, Q, K, or H
  MOD_RES (7)..(7): Y, G, S, D, T, R, A, L, V, P, N, F, E, I, W, Q, K, or H
  MOD_RES (8)..(8): Y, G, S, D, T, R, A, L, V, P, N, F, E, I, W, Q, K, or H
GXIXXXXXM

SEQ ID NO: 91 - protein, 14 aa, Artificial Sequence
  Description of Artificial Sequence Synthetic peptide
  MOD_RES (2)..(2): F or L
  MOD_RES (5)..(5): A, G, S, or T
  MOD_RES (7)..(7): A, D, G, N, S, or T
  MOD_RES (8)..(8): Y, G, S, D, T, R, A, L, V, P, N, F, E, I, W, Q, K, or H
  MOD_RES (10)..(10): A, G, S, or T
  MOD_RES (11)..(11): I, N, S, T
  MOD_RES (13)..(13): N or Y
EXVAXIXXGXXTXY

SEQ ID NO: 92 - protein, 13 aa, Artificial Sequence
  Description of Artificial Sequence Synthetic peptide
  MOD_RES (2)..(2): F or L
  MOD_RES (6)..(6): A, G, S, or T
  MOD_RES (7)..(7): Y, G, S, D, T, R, A, L, V, P, N, F, E, I, W, Q, K, or H
  MOD_RES (9)..(9): A, G, S, or T
  MOD_RES (10)..(10): I, N, S, T
  MOD_RES (12)..(12): N or Y
EXVAIXXGXXTXY

SEQ ID NO: 93 - protein, 126 aa, Artificial Sequence
  Description of Artificial Sequence Synthetic polypeptide
QVQLLESGGGLVKPGGSLRLSCAASGFTFSDYYMSWIRQAPGKGLEWVAGINYGSNTYYADSVKGRFTISRDNAKNSLYLQMNSLRAEDTAVYYCAASQRRAYAARIDLFSYHTYGGQGTLVTVSS

SEQ ID NO: 94 - protein, 126 aa, Artificial Sequence
  Description of Artificial Sequence Synthetic polypeptide
QVQLLESGGGLVKPGGSLRLSCAASGFTFSDYYMSWIRQAPGKGLEWVAGINYGSNTYYADSVKGRFTISRDNAKNSLYLQMNSLRAEDTAVYYCAASQRRAYAARIDLFSYHTYWGQGTLVTVSS

SEQ ID NO: 95 - protein, 115 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPCQQSYTSPRTFGQGTKVEIK

SEQ ID NO: 96 - protein, 115 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPCQQTYTTPRTFGQGTKVEIK